(19) United States
(12) Patent Application Publication
(10) Pub. No.: US 2025/0078969 A1
(43) Pub. Date: Mar. 6, 2025

(54) SYSTEMS AND METHODS FOR AUTOMATED EVIDENCE GENERATION

(71) Applicant: ATROPOS HEALTH, INC., New York, NY (US)

(72) Inventors: Yen Sia Low, San Francisco, CA (US); Rehan N. Chawdry, Dublin, OH (US); Robert Edison Brown, Los Angeles, CA (US); Saurabh Gombar, San Diego, CA (US); Neil M. Sanghavi, Dublin, OH (US)

(21) Appl. No.: 18/816,493

(22) Filed: Aug. 27, 2024

Related U.S. Application Data
(60) Provisional application No. 63/579,499, filed on Aug. 29, 2023.

(51) Int. Cl. G16H 10/60; G06F 40/166; G06F 40/237

(52) U.S. Cl. CPC G16H 10/60; G06F 40/166; G06F 40/237

(57) ABSTRACT

Some embodiments relate to receiving a natural language question for a study, such as a retrospective study. Some embodiments include formatting the question according to a defined question format. Some embodiments include utilizing a phenotype library to map information provided in the question to specific medical codings. Some embodiments include retrieving patient health record data and performing automated statistical analysis. Some embodiments include summarizing study data. Some embodiments include identifying and summarizing relevant published articles.

### INCORPORATION BY REFERENCE TO ANY PRIORITY APPLICATIONS

Any and all applications for which a foreign or domestic priority claim is identified in the Application Data Sheet as filed with the present application are hereby incorporated by reference under 37 CFR 1.57. This application claims the benefit of priority to U.S. Provisional Application No. 63/579,499, filed Aug. 29, 2023, the contents of which are hereby incorporated by reference in its entirety and for all purposes as if set forth fully herein.

### BACKGROUND

### Field

This disclosure relates to systems and methods for automated clinical decision guidance.

### Description

The approaches described in this section are approaches that could be pursued, but not necessarily approaches that have been previously conceived or pursued. Thus, unless otherwise indicated, it should not be assumed that any of the material described in this section qualifies as prior art merely by virtue of its inclusion in this section.

Physicians, researchers, and other health professionals face many challenges when determining appropriate treatments, evaluating the efficacy of treatments, and so forth. While there can be a large amount of data that could be used to inform decisions, making use of that data remains challenging. Accordingly, there is a need for improved systems and methods for determining answers to questions based on health information.

### SUMMARY

For purposes of this summary, certain aspects, advantages, and novel features are described herein. It is to be understood that not necessarily all such advantages may be achieved in accordance with any particular embodiment. Thus, for example, those skilled in the art will recognize the disclosures herein may be embodied or carried out in a manner that achieves one or more advantages taught herein without necessarily achieving other advantages as may be taught or suggested herein.

All of the embodiments described herein are intended to be within the scope of the present disclosure. These and other embodiments will be readily apparent to those skilled in the art from the following detailed description, having reference to the attached figures. The invention is not intended to be limited to any particular disclosed embodiment or embodiments.

In some aspects, systems and methods herein can enable the automated analysis of electronic health records, published studies, or both. In some aspects, analyses can be used for evidence-driven clinical guidance.

In some embodiments, the techniques described herein relate to a computer-implemented method for performing a retrospective medical study including: receiving an input from a user via a user interface, wherein the input includes a natural language question, wherein the input describes a medical research question; processing the input to determine a study type for the input; determining, based on the input, one or more study parameters, wherein determining the one or more study parameters is done at least in part by providing the input to a first large language model; generating, using the first large language model, a formatted study question based at least in part on the study type and the one or more study parameters; processing the formatted study question to determine a phenotype associated with the formatted study question; generating, based at least in part on the formatted study question and the phenotype, a database query; executing the database query against a database to retrieve health record data for patients matching the one or more study parameters; analyzing the retrieved health record data based on a predefined analysis procedure; generating a summary of the analyzed health record data using a second large language model; and making the summary available to the user.

In some embodiments, the techniques described herein relate to a computer-implemented method, wherein processing the input to determine the study type includes identifying a specification of a study type in the input.

In some embodiments, the techniques described herein relate to a computer-implemented method, wherein processing the input to determine the study type includes: generating a prompt for the first large language model, the prompt including at least a portion of the input and an instruction to determine the study type; and submitting the prompt to the first large language model; and receiving a response, the response including the study type.

In some embodiments, the techniques described herein relate to a computer-implemented method, further including: identifying a publication related to the formatted study question; accessing, over a communications network, an electronic version of the publication; generating a summary of the publication using a third large language model; and making the summary available to the user.

In some embodiments, the techniques described herein relate to a computer-implemented method, wherein the second large language model is the same as the first large language model.

In some embodiments, the techniques described herein relate to a computer-implemented method, wherein the first large language model is configured to use retrieval augmented generation to generate the formatted study question, wherein generating the formatted study question includes: generating an embedding of the input; comparing the embedding of the input to a plurality of embeddings in a knowledge base; identifying, based on the comparison, a set of relevant reference documents; augmenting the input with contextual information derived from at least one of reference documents in the set of relevant reference documents; and providing the augmented input to the first large language model.

In some embodiments, the techniques described herein relate to a computer-implemented method, wherein the input is augmented with a subset of the set of relevant documents, wherein the subset is determined by evaluating each reference document of the set of reference documents, wherein the evaluation is based at least in part on one or more of: relevance, source credibility, or publication date.

In some embodiments, the techniques described herein relate to a computer-implemented method, wherein the second large language model is configured to use retrieval augmented generation.

In some embodiments, the techniques described herein relate to a computer-implemented method, wherein determining the phenotype includes: identifying a term in the input indicating a condition, procedure, or medication; querying a phenotype library using the identified term to identify a corresponding record; and identifying one or more codes associated with the term.

In some embodiments, the techniques described herein relate to a computer-implemented method, further including, after generating the formatted study question: providing the formatted study question to the user; receiving a request from the user to revise the formatted study question; and revising the formatted study question, wherein revising the study question including modifying at least one of the one or more study parameters.

In some embodiments, the techniques described herein relate to a computer-implemented method, further including, after generating the formatted study question: providing the study question to the user; receiving a request to edit the formatted study question from the user; receiving one or more user edits to the formatted study question; and updating the formatted study question based on the one or more user edits.

In some embodiments, the techniques described herein relate to a computer-implemented method, wherein determining the phenotype includes: extracting a term from the input; generate a prompt for a fourth large language model using the extracted term, the prompt configured to cause the fourth large language model to identify a closest matching phenotype; provide the prompt to the fourth large language model; and receive an output from the fourth large language model responsive to the prompt; and identify the phenotype based on the received output.

In some embodiments, the techniques described herein relate to a computer-implemented method, wherein analyzing the retrieved health record data based on the predefined analysis procedure includes: identifying a study template associated with the determined study type, wherein the study template includes one or more variable parameters; updating at least one of the one or more variable parameters of the study template based on at least one of: the input, the one or more study parameters, or the phenotype; and processing the updated study template to determine one or more variables of interest using the retrieved health record data.

In some embodiments, the techniques described herein relate to a computer-implemented method, wherein analyzing the retrieved health record data includes: determining a set of preliminary results; providing the set of preliminary results to the user; receiving a confirmation from the user to continue the analysis; and in response to receiving the confirmation from the use, continuing the analysis of the retrieved health record data.

In some embodiments, the techniques described herein relate to a computer-implemented method, wherein the preliminary results include at least one of: number of a patients included in the health record data, age ranges of patients included in the health record data, number of patients receiving a particular treatment, or gender distribution of patients included in the health record data.

### BRIEF DESCRIPTION OF THE DRAWINGS

These and other features, aspects, and advantages of the disclosure are described with reference to drawings of certain embodiments, which are intended to illustrate, but not to limit, the present disclosure. It is to be understood that the accompanying drawings, which are incorporated in and constitute a part of this specification, are for the purpose of illustrating concepts disclosed herein and may not be to scale.

FIG. 1 is a flowchart that illustrates an example process for performing a retrospective study based on a user input according to some embodiments.

FIG. 2 is a flowchart that illustrates another example process for performing a retrospective study according to some embodiments.

FIG. 3 is a flowchart that illustrates an example process that can be carried out on a computer system to generate clinically relevant information for a user.

FIG. 4 is a diagram that schematically illustrates an example user interface that can be used for question generation according to some embodiments.

FIG. 5 is a flowchart that illustrates an example process for recommending changes to a formatted question according to some embodiments.

FIG. 6 is a flowchart that illustrates an example process for phenotyping according to some embodiments.

FIG. 7 illustrates an example of a process for training a machine learning model to generate phenotype library entries according to some embodiments.

FIG. 8 illustrates an example process for generating phenotype library entries according to some embodiments.

FIG. 9 is a flowchart that illustrates another example process for generating phenotypes according to some embodiments.

FIG. 10 illustrates an example process for generating a query according to some embodiments.

FIG. 11 is a flowchart that illustrates an example process for generating a query according to some embodiments.

FIG. 12 is a flowchart that illustrates an example process for generating a query according to some embodiments.

FIG. 13 is a flowchart that illustrates an example process for generating and summarizing statistical data according to some embodiments.

FIG. 14 is a flowchart that illustrates an example process for summarizing published articles according to some embodiments.

FIG. 15 is a schematic that illustrates an example of a phenotype database record and user interface for viewing phenotype database records according to some embodiments.

FIG. 16 is a flowchart that illustrates an example process for generated augmented queries using retrieval augmented generation according to some embodiments.

FIG. 17 depicts a process for training an artificial intelligence or machine learning model according to some embodiments.

FIG. 18 is a block diagram depicting an embodiment of a computer hardware system configured to run software for implementing one or more embodiments disclosed herein.

### DETAILED DESCRIPTION

Although several embodiments, examples, and illustrations are disclosed below, it will be understood by those of ordinary skill in the art that the inventions described herein extend beyond the specifically disclosed embodiments, examples, and illustrations and includes other uses of the inventions and obvious modifications and equivalents thereof. Embodiments of the inventions are described with reference to the accompanying figures, wherein like numerals refer to like elements throughout. The terminology used in the description presented herein is not intended to be interpreted in any limited or restrictive manner simply because it is being used in conjunction with a detailed description of certain specific embodiments of the inventions. In addition, embodiments of the inventions can comprise several novel features and no single feature is solely responsible for its desirable attributes or is essential to practicing the inventions herein described. The terms “model,” “machine learning model,” and similar terminology as used herein can refer to a construct that can be trained using training data to make predictions, to provide probabilities related to new data, to generate new data (e.g., new text, new images, summaries, etc.), and so forth.

### Overview

Retrospective studies are a powerful tool for medicine and can provide insights that can be used to improve care for groups ranging from entire populations to individual patients. However, there are many difficulties associated with retrospective studies, which can limit their use. For example, obtaining and analyzing data can be challenging, confounding variables can make it difficult to draw meaningful inferences, and so forth. The systems and methods described herein can enable physicians and other medical professionals to more easily make use of retrospective studies.

Health professionals such as physicians, clinical researchers, pharmaceutical researchers, and so forth often struggle to make sense of large volumes of clinical data, claims data, published research papers, and so forth. Practicing physicians may find themselves especially constrained as they may be busy seeing patients and have little time to devote to researching questions. Even researchers who are focused on conducting research may nonetheless find it tedious and time consuming to analyze health data, and the significant effort that may be put in can reduce the amount of research a health professional does, the depth or thoroughness of research, and so forth. In some cases, a researcher may be unaware of certain features (e.g., correlations, causal relationships, etc.) in available data, and may have no practical way of uncovering such features when there are complex relationships between different variables included in the data.

When a health professional wishes to investigate a question, doing so can be a long and difficult process. For example, as described herein, finding data (e.g., electronic health record (EHR) data, claims data), published papers, analyzing data, etc., can involve a considerable time commitment and may be outside the skillset of many medical professionals. As an example, a health professional who wishes to conduct a retrospective observational study (e.g., a study of past patients) can formulate a question, identify relevant patient populations, retrieve relevant data, perform statistical analysis of the data, and interpret the results. Each of these steps can present significant challenges.

For example, formulating a question can be difficult for a variety of reasons. For example, it can be important to determine an appropriate age group, control conditions (e.g., no treatment, standard treatment, etc.), timeframes, and so forth. Identifying the patient population can be a significant undertaking. For example, a physician who wishes to perform analysis of patients with diabetes may search for patients whose health records include particular codes (e.g., ICD-10 codes) that correspond to diabetes. Data may come from a variety of sources, different coding schemes may be used, there may be many different codes that relate to diabetes, and so forth, which can make it difficult to identify relevant patients. A researcher may not be aware of all the relevant diagnostic codes, procedure codes, and so forth, and thus may miss certain patients who should be included in the study. After determining relevant patients, additional challenges remain. Retrieving data can be difficult, as the data may be stored in different databases, have different formats and/or field names, and so forth.

Aggregating data from diverse sources can prove challenging as it may be important to map fields to one another, convert formats, convert units (e.g., temperature, weight, etc.), and so forth. After retrieving and preparing the data, the health professional can then perform statistical analysis on the data. Such analyses can involve a significant effort and are prone to user error. After conducting statistical analysis, the health professional can review the results and determine insights provided by the results (e.g., the health professional may observe that a first intervention performs better than a second intervention in a first population, but the reverse is true in a second population). Each of these steps can involve significant effort and require certain expertise. For example, a physician may be unfamiliar with how to transform data, how to write queries (which can be especially complicated when data is stored in different systems, across multiple tables, etc.), how to perform statistical analysis, how to interpret statistical results, and so forth. In some cases, performing such work may only be feasible for a limited set of health professionals, such as those affiliated with major pharmaceutical companies, medical schools, research institutions, etc., where they have access to experts in areas such as data science. Yet, other healthcare professionals and their patients could obtain substantial benefits from being able to conduct data-driven investigations into prognoses, disease treatment, and so forth. Moreover, even dedicated researchers can benefit from thorough and accurate population definition, data analysis, and so forth. As described herein, there are many benefits to an analytical platform beyond retrieving and analyzing data (which are substantial benefits in their own right and as described in more detail herein). These additional benefits are described in more detail herein.

According to some embodiments of the present disclosure, a platform facilitates providing clinically relevant information to physicians, researchers, and other health professionals based on health record data, published papers, or both. Using the systems and methods herein, users can pose questions and receive statistically sound, clinically relevant responses to those questions.

While retrospective studies can be a powerful tool, there are significant drawbacks and difficulties associated with retrospective studies. One major issue is selection bias, as retrospective studies typically rely on existing records that may not represent the entire population. Recall bias is another problem, in that retrospective studies can rely on potentially inaccurate recollection of past events. EHR records, claims records, and so forth can be incomplete or contain incorrect information, which can limit a study's ability to draw accurate conclusions. The quality of data used in studies can vary significantly, with records potentially being inaccurate, inconsistent, recorded using different methods, etc. A researcher or physician making use of such data has no control over how the data was originally collected. Confounding variables can be difficult to control and can influence the apparent relationships between inputs and outputs (e.g., between a treatment and a medical outcome). Thus, it can be important to take measures the ensure that data is of a suitable quality, volume, etc., before relying on results obtained using the data. In some embodiments, a platform as described herein can guard against such issues by analyzing data to identify and address such issues, such as by excluding certain patients, refining a researcher's question, and so forth.

Retrieving and analyzing large volumes of patient data can be computationally intensive, requiring significant computing resources, time, and energy to complete. In some cases, the results of a retrospective study may not be suitable for use, for example because the underlying data does not have adequate information to answer a question under investigation, or there are not enough patients that match study criteria to obtain statistically significant insights. In some cases, it may be worthwhile to continue with a study even when the results of such a study may have limited probative value. For example, such a study can still be useful for indicating whether further investigation is warranted. In other cases, a researcher may want to abandon a study so that the study question can be modified to produce more statistically reliable and/or clinical relevant results.

Accordingly, it can be beneficial to provide preliminary results before running a full analysis or simultaneously with running a full analysis. This can help prevent wasting time and resources analyzing data that will not or is unlikely to lead to actionable insights. In some embodiments, the systems and methods herein can provide preliminary information and/or preliminary results. That is, before performing a full analysis, or concurrently with performing a full analysis, the systems and methods herein can provide preliminary information that can be used by a researcher to determine whether or not to continue the study. In some embodiments, a system can be configured to automatically determine whether or not to continue a study, for example, based on the number of patients included in the study, the presence or number of confounding variables, and so forth.

There are many different types of retrospective studies, some of which are described herein. Regardless of the study type, it can be significant to carefully prepare a study question to ensure that the study is well-defined and key study criteria are not missing or incomplete. There are various formats for defining retrospective studies. One example is PICOT, which has emerged as a framework in the medical field for formulating research questions. PICOT can provide a structured approach that helps ensure relevant aspects of a medical issue are considered, which can lead to better insights. PICOT is an acronym that stands for population (or in some cases, patient), intervention, comparison, outcome, and time.

Population specifies a population (e.g., group of patients) to be studied. Population can include information such as age, gender, ethnicity, specific health conditions, etc. Intervention can identify a specific treatment, procedure, action, etc., being investigated. For example, an intervention can be a drug, surgical technique, lifestyle change, physical or mental therapy, or any other healthcare intervention. Comparison involves comparing the intervention with another treatment or a control group. The comparison can include comparing an intervention group (e.g., a new treatment) with a control group (for example, a current standard treatment, no treatment, etc.). Outcome can specify desired results of the intervention. Outcomes can include clinical measures such as blood sugar levels, blood pressure, quality of life, healthcare cost, etc. Time can define a duration over which the intervention's effects are measured. Depending on the study, the time could be, for example, days, weeks, months, or years.

While PICOT is a common framework for formulating medical research questions, other approaches are possible and may be more readily applicable to different types of questions. For example, PECO (Participants, Exposure, Comparison, Outcome) can omit the “intervention” element and can be more suited to observational studies that examine the effect of a certain exposure (e.g., excess sun exposure, exposure to a toxin, exposure to fatty or sugary food, etc.) without a specific intervention being introduced. SPIDER (Sample, Phenomenon of Interest, Design, Evaluation, Research Type) can include more detail than PICOT. For example, SPIDER includes a defined study type (e.g., qualitative interviews, quantitative surveys, qualitative evaluation of patient records, quantitative evaluation of patient records (e.g., lab results), etc.), the type of research being conducted, and so forth. SPICE (Setting, Perspective, Intervention, Comparison, Evaluation) can include information about the specific setting where the research takes place (e.g., rural vs. urban hospitals), evaluation methods, etc. SPICE can be used to design studies from the perspective of various stakeholders, such as achieving the best patient outcomes, utilizing available resources (e.g., clinical staff or budgets) effectively, etc. ECLIPSE (Explanation, Context, Literature, Intervention, Policy, Evaluation) is yet another framework that is often used in health policy research. ECLIPSE can be well-suited to exploring the broader context behind a particular health issue, taking into consideration relevant research, potential policy implications, and so forth. As an example, ECLIPSE could be used to investigate the impact of banning smoking indoors, extending bar hours from 2 AM to 4 AM, building more public playgrounds, and so forth.

Studies can be divided broadly into several categories, such as observational studies, experimental studies, and case series studies. Observational studies involve observing and collecting data on patients without directly influencing their health or behavior. Observational studies can be broken down into various types, such as cohort studies, in which a group is tracked over time, case-control studies, in which one group of patients is compared to another group of patients that is generally similar but differs in at least one aspect under investigation. For example, a case-control study could compare otherwise similar patients, one group of which is affected by a particular condition or provided with a particular treatment while the other group is not. Cross-sectional studies can be used to capture a snapshot in time that can capture associations between various factors (e.g., diet, exercise, etc.) and disease prevalence.

Case series studies are a type of observational study that typically focuses on a small group of individuals who have a particular condition or have received a specific treatment. Case series can lack the statistical power of larger studies, but can be valuable in certain contexts. For example, case series can be used to investigate rare conditions, potentially providing insight into the characteristics, causes, or both of such conditions, which can help enable further investigation, development of diagnostic criteria, etc. In some cases, case series are used for initial testing of new treatments. In such cases, case series data can provide preliminary data regarding safety and efficacy of a potential treatment. Case series can also be used to generate hypotheses about potential causes, treatment targets, etc.

The approaches described herein can be used to carry out studies of various types in a sound manner that does not rely on researcher expertise in coding, statistics, databases, etc. For example, in some embodiments, a researcher can pose a question in plain language terms, and a platform can process the natural language question, for example using a large language model (LLM) to generate a study question. In some embodiments, the platform can automatically suggest improvements to the researcher's question, automatically perform data analysis, identify and summarize relevant research papers, and so forth. These and other aspects are described in more detail below with reference to the figures.

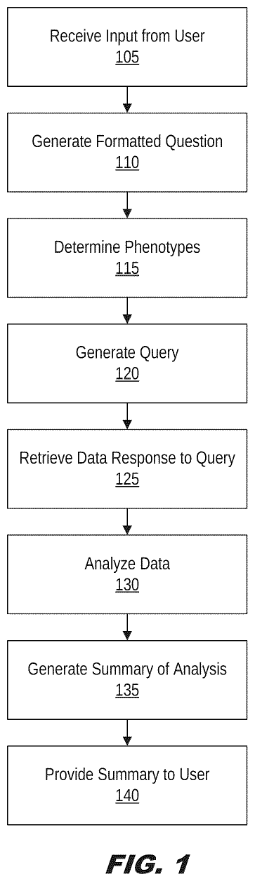

FIG. 1 is a flowchart that illustrates an example process for performing a retrospective study based on a user input according to some embodiments. At operation 105, a system can receive an input from a user. The input can be, for example, a natural language question. At operation 110, the system can, based on the input, generate a formatted question. For example, the system can provide the input as part of a prompt to an LLM, and the LLM can generate a formatted question. As an example, a prompt can include the user's input and a specification of what type of question to generate.

In some embodiments, the system can receive a user selection of a type of formatted question to generate. In some embodiments, the system can determine the type of formatted question based on inputs received from the user. For example, a user may not specify a particular study type, but the study type can be inferred by the LLM based on the input provided by the user.

At operation 115, the system can determine one or more phenotypes associated with the question. For example, if a user enters “diabetes,” the system can determine one or more phenotypes associated with diabetes. The phenotypes can indicate diagnostic codes, procedure codes, medication codes, etc., associated with diabetes. At operation 120, the system can generate a query. In some embodiments, the system can generate the query using a template. For example, a query template can include variables where information from the formatted question, the determined phenotypes, etc., can be substituted in. This can help to ensure that the query will execute and will retrieve the information needed for analysis. At operation 125, the system can retrieve data responsive to the query. For example, the system can query a database or other data store to retrieve data relevant to the query. At operation 130, the system can analyze the data. For example, in some embodiments, the system can analyze the data to generate a set of statistics. At operation 135, the system can generate a summary of the analysis. In some embodiments, the system can utilize an LLM to generate the summary. For example, the system can provide the LLM with the calculated statistics and instructions for how to generate the summary (e.g., writing style, length, statistics to include, etc.). In some embodiments, the system can provide the LLM with an example summary. At operation 140, the system can provide the summary to the user, for example as an HTML page or PDF.

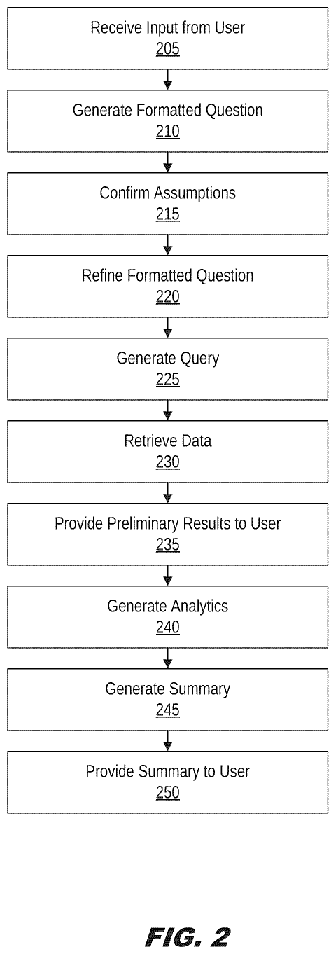

FIG. 2 is a flowchart that illustrates another example process for performing a retrospective study according to some embodiments. At operation 205, a system can receive an input from a user. The input can be, for example, a research question. At operation 210, the system can generate a formatted question, for example using an LLM. When generating the formatted question, the system can make one or more assumptions, such as assumptions about age range, time period, and so forth. At operation 215, the system can confirm the assumptions. For example, the system can cause the assumptions to be displayed to the user, and the user can confirm that the assumptions are acceptable. In some embodiments, the user can edit assumptions. At operation 220, the system can refine the formatted question, for example in response to a user request to improve the formatted question. For example, the system can suggest adding or removing a condition or treatment, adjusting an age range, etc. In some embodiments, the recommendations can be based on past clinical trials, information about a number of patients who match the study criteria, etc. For example, if a user specifies a narrow age range or a short time period, the system can determine that there are few patients who match the study criteria and can suggest modifying the study criteria to include more patients. In some embodiments, the system refines the question using an LLM. In some embodiments, the system is configured to utilize retrieval augmented generation, for example as described herein, to provide context for refining the question, which can help to ensure that suggested refinements are clinically meaningful and/or sensible. In some embodiments, the system refines the study in response to a user request to improve or otherwise modify the study. At operation 225, the system can generate a query based on the question. In some embodiments, the system generates the query using a template. The template can be configured for a particular study type. In some embodiments, a template may not be used. For example, an LLM can be instructed to generate a query. In some embodiments, an LLM prompt can include information such as a database schema to use when generating the query. At operation 230, the system can execute the query and retrieve data matching the query. At operation 235, the system can provide preliminary results to the user. For example, the system can provide information to the user such as the number of patients included in the study, age ranges of patients included in the study, gender distribution of patients included in the study, number of patients receiving a particular treatment, or other preliminary results. In some embodiments, the user can provide input to the system indicating whether or not to continue the analysis. For example, a user may observe that the preliminary results contain too few patients or otherwise are unlikely to produce the desired results and can instruct the system to abort further processing. This can be significant as statistical calculations can be computationally intensive. At operation 240, the system can perform further analysis of the data. In some embodiments, operation 240 can be carried out concurrently with operation 235, such that analysis operations continue while the user reviews the preliminary results or continues even if the user does not review the preliminary results. In some embodiments, operations 235 and 240 can be carried out in series, such that analysis does not continue without user confirmation. At operation 245, the system can generate a summary of the analytics. At operation 250, the system can provide the summary to the user.

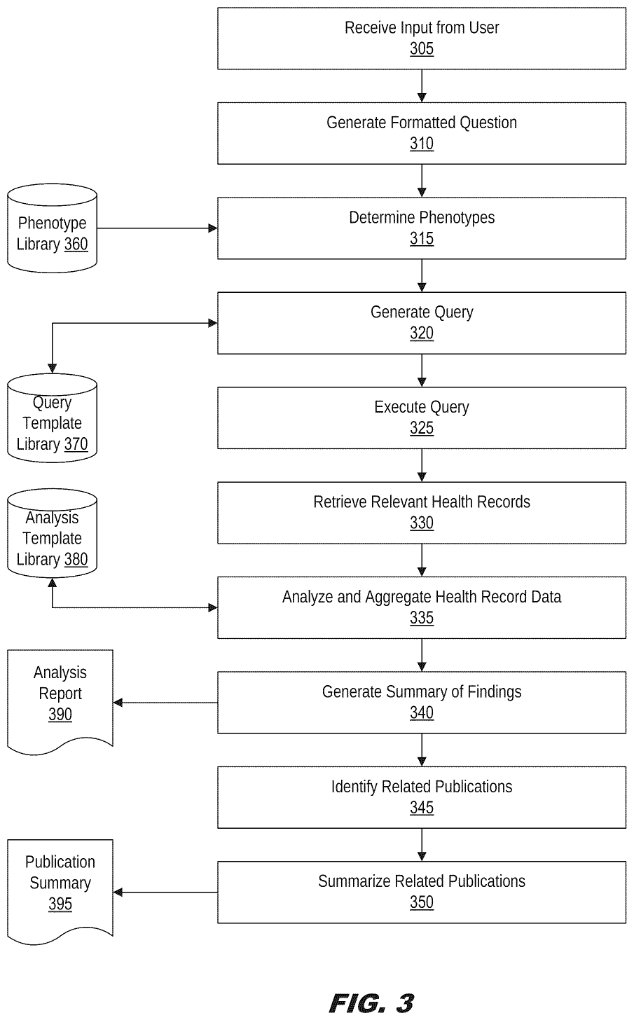

FIG. 3 is a flowchart that illustrates an example process that can be carried out on a computer system to generate clinically relevant information for a user.

At operation 305, the system can receive an input from a user. In some embodiments, the input can be a natural language question. At operation 310, the system can generate a formatted question that can be a representation of the question received from the user. At operation 315, the system can determine phenotypes associated with the formatted question. The system may make use of a phenotype library 360. The phenotype library 360 can be a database or other data store that includes information that can be used to determine relevant health record data based on the formatted question. For example, the phenotype library can map a term (e.g., “hypertension” to diagnostic codes associated with hypertension). There can be various coding schemes used in health record data, such as International Classification of Diseases (ICD), Logical Observations Identifiers Names and Codes (LOINC), Systematic Nomenclature of Medicine (SNOMED), Current Procedural Terminology (CPT), and so forth. Approaches for creating and using a phenotype library are described in more detail herein.

At operation 320, the system can generate a query (e.g., an SQL query, MUMPs query, etc.) to retrieve data from a database of electronic health records. The query can be generated in any suitable language, which can include a custom language. In some embodiments, a database and/or query language can be optimized for retrieving and analyzing time-series data, such as health record data collected across various patient encounters. In some embodiments, data can be stored in an in-memory database to enable faster retrieval. In some embodiments, the in-memory database is a time-series database. In some embodiments, the system can use a query template library 370 as a basis for generating a query. For example, in some embodiments, a study design can be specified by a user or inferred from the formatted question, and a query template corresponding to the study design can be used for generating a query. For example, in some embodiments, a template may include named fields that correspond to PICOT fields. In some embodiments, the query can incorporate information from the phenotype library. For example, if a user entered hypertension in a question prompt, the generated query may search for health records that include ICD-10 codes I10-I16, as determined by looking up hypertension in the phenotype library. In some embodiments, a query template may not be used.

At operation 325, the system can execute the generated query, and, at operation 330, the system can retrieve relevant health records responsive to the query. At operation 335, the system can analyze and aggregate the retrieved health record data. In some embodiments, the system can utilize a template in an analysis template library 380. The template can define, for example, certain types of analyses to be conducted. At operation 340, the system can generate a summary of findings of the analysis, the formatted question, etc. The analysis results at operation 335, the summary at operation 340, both, and/or additional information can be included in a statistical analysis report 390. At operation 345, the system can retrieve and summarize related publications, such as peer-reviewed, published studies. For example, the system can identify relevant publication based on the formatted question, the received question, etc. The summaries can be included in a publication summary 395. The publication summary 395 can be a part of the analysis report 390 in some embodiments.

### Question Generation

Healthcare providers, clinical researchers, pharmaceutical researchers, and other health professionals may have a question in mind that they would like to investigate. However, they may not have formulated the question in a manner that is conducive to conducting an automated analysis of EHR data, claims data, and/or other types of health record data. For example, there may be missing or incomplete information, improper formatting, or other issues with a question. Thus, it can be significant to receive a question and to use the content of the question to generate a question that adheres to a particular format, for example a well-formulated PICOT question.

In some embodiments, a user can submit a natural language question, and a system can be configured to output a formatted representation of the question. In some embodiments, the system can fill in missing information with default information. In some embodiments, the system can flag missing information or information that was filled in with default values so that the user can review the missing or default information. For example, a user may omit a time period, age range, control group, and/or other information, and the system may recommend defaults for such missing information. In some embodiments, the system can recommend defaults based on values used in other studies. For example, the system can access prior studies and identify similar studies (e.g., similar populations, similar treatments, etc.), which can be used in determining one or more default values.

In some embodiments, default values can be supplied when information is missing. For example, if age is not specified, the system can be configured to supply a default value, such as all adults aged eighteen and over. In some embodiments, the system may be configured to infer that a question pertains to a particular age range, in which case that age range may be used as a default age range. For example, if a question is about childhood diabetes, the system may automatically set a default age range that includes minors and does not include adults. As another example, a health professional may omit a timeframe. In some embodiments, the system can supply a default timeframe, such as the last year, the last five years, the last ten years, since 2010, since 2015, all available data, etc. In some cases, the system can infer a date based on a condition. For example, if a question relates to COVID-19, the system may limit the time range for health record data to 2019 or later, since there would be no diagnoses, treatments, lab results, etc., prior to 2019. In some embodiments, the system can infer a time period based on provided information, such as a provided condition. For example, if a physician asks a question related to Alzheimer's disease, the system can determine an age range that covers middle-aged individuals through death. In some cases, a health professional may omit a control. For example, a physician may pose a question about the effectiveness of a particular antibiotic for a specified condition but may not specify a control group. In some embodiments, the system can provide a default control group, for example “patients who did not receive the particular antibiotic,” “patients who received a different antibiotic,” or “patients who received a standard treatment regimen.” Information such as other antibiotics, standard treatment regimens, etc., can be determined in various ways, such as by querying a phenotype library or using retrieval augmented generation (RAG). For example, RAG can be used to provide contextual information such as standard treatments published in journals, on official web sites, and so forth.

As described herein, parameters or values that are supplied by the system can be indicated to the submitter so that the submitter (or an intermediary) can ensure that the final question is suitable. Continuing with the example above, it will be appreciated that a comparison of the particular antibiotic to no antibiotic treatment may produce a very different result than a comparison of the particular antibiotics to other antibiotics that are used to treat the specified condition.

In some embodiments, a system may be configured to use a large language model to generate questions in a particular format, such as PICOT. As an example, a physician or other health professional may ask a question such as “What is the average improvement in systolic blood pressure in patients with hypertension treated with ACE inhibitors?” In a PICOT format, the question could be framed as, for example, “Population: Hypertension patients; Intervention: ACE inhibitors; Control: No treatment with ACE inhibitors; Outcome: change in systolic blood pressure; Timeframe: Not specified.” In this example, while there was no control group specified in the original question, the system has supplied a default control group of hypertension patients who did not receive the treatment under investigation.

In some embodiments, a system can be configured to suggest improvements to the study. For example, the system may be configured to recommend changes to treatments, age groups, control conditions, comparison conditions, timeframes, etc. In some embodiments, a phenotype library, as discussed in more detail herein, can be used to suggest possible improvements to a study. Other sources of information may also be used, additionally or alternatively. For example, if a user specifies treatment with cefoxitin in a prompt, the system may use the phenotype library to determine classes, sub-classes, etc., of drugs to which cefoxitin belongs. For example, the system may suggest including all second-generation cephalosporins, all cephalosporins, etc. As another example, if a user specifies an age range, the system can suggest a different age range (e.g., a narrow age range or a broader age range), a developmental stage, etc.

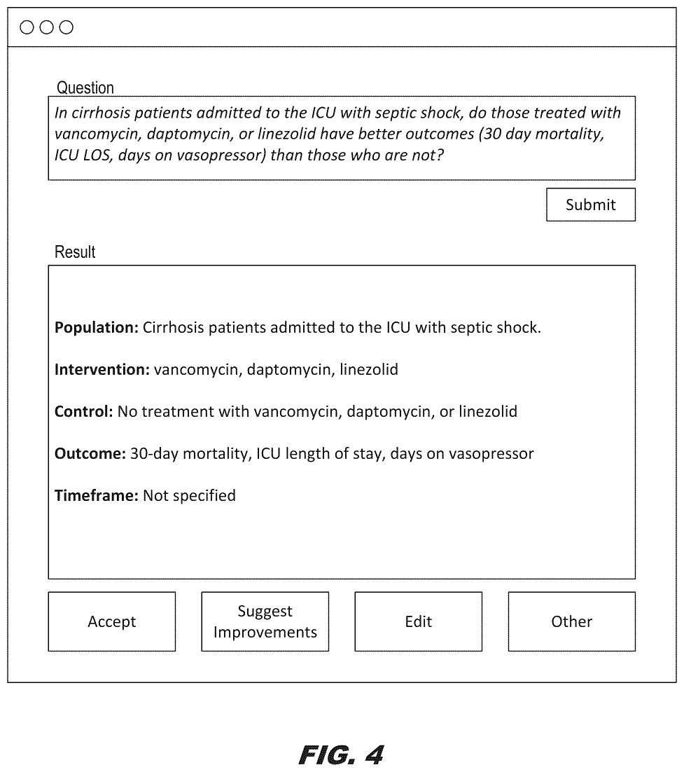

FIG. 4 is a diagram that schematically illustrates an example user interface that can be used for question generation according to some embodiments. The user interface can include an input box in which a user can enter a prompt to be submitted to a large language model (LLM) to generate a PICOT or other formatted study question or definition. In some embodiments, the user can enter a natural language question. The user interface can include a button for the user to submit the prompt to the LLM. In some embodiments, the user may, alternatively or additionally, use a keyboard shortcut to submit the prompt (e.g., return, enter, etc.). The user interface can include a result section that displays the generated formatted question. The user interface can include one or more buttons or other UI elements. In some embodiments, a user interface can include buttons that the user can click to accept the generated formatted question, request improvements to the formatted question, manually edit the formatted question or perform other actions.

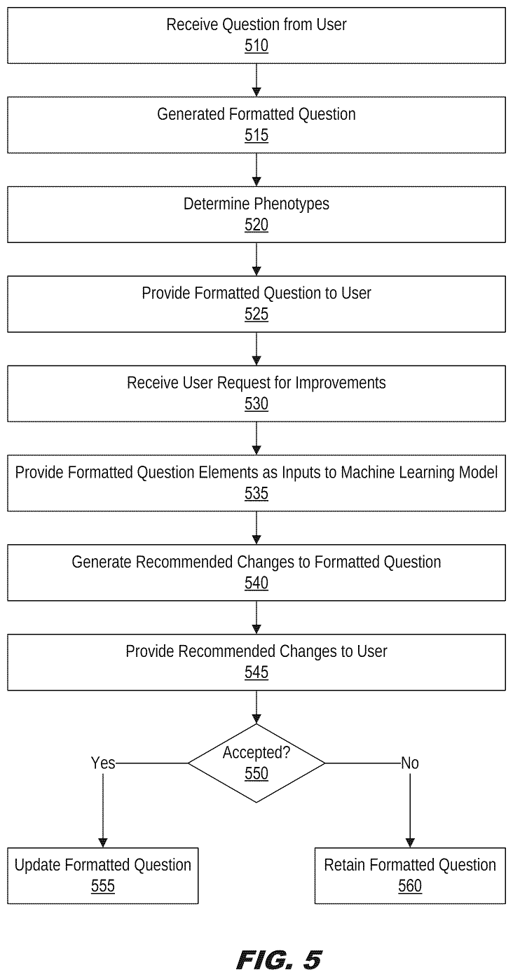

FIG. 5 is a flowchart that illustrates an example process for recommending changes to a formatted question according to some embodiments. The process depicted in FIG. 5 can be performed by a computing system.

At operation 510, the system can receive a question from a user. The question can, for example, be an LLM prompt, a natural language question, etc. At operation 515, the system can, using an LLM, generate a formatted question based on the question submitted by the user. At operation 520, the system can determine phenotypes, for example using a phenotype library as described herein. At operation 525, the system can present the formatted question to the user. At operation 530, the system can receive a user request for suggested improvements. At operation 535, the system can provide the formatted question or elements of the formatted question as inputs to a machine learning model, such as an LLM. At operation 540, the model can generate recommended changes to the formatted question. At operation 545, the system can provide recommended changes to the user. At decision 550, the user can accept or reject the recommendations. If the user accepts the recommendations, the system can update the formatted question at operation 555. If the user rejects the recommendations, the system can retain the original formatted question at operation 560. In some embodiments, the system can provide multiple recommendations in response to multiple user requests for recommendations.

In some embodiments, recommendations can be based on information about prior studies, prior published articles, and so forth. In some embodiments, the system can use RAG to provide contextual information to an LLM, which can improve recommendations made by the LLM.

### Phenotypes

Phenotypes can play an important role in retrospective studies. As described herein, it can be difficult to identify all the patients who should be included in a study. Phenotypes can refer to the observable characteristics of patients. Phenotypes can map conditions or other characteristics to specific codes or characteristics. As an example, if a user enters a question requesting information about male patients over 65 with type II diabetes, a phenotype can be used to determine that type II diabetes should map to particular ICD codes, particular lab results (e.g., high A1C lab results), and so forth. Thus, the physician or other researcher does not need to know the precise codes, lab results, etc., that indicate a particular condition, but can instead use natural language description which can then be used to determine the relevant codes, lab results, etc., to identify patients who should be included in the study.

Phenotypes can also be used to describe relationships between treatments, medications, etc. For example, a phenotype can include a class of antibiotics, type of anti-depressant (e.g., all selective serotonin reuptake inhibitors, etc.).

As discussed above, a condition, treatment, test, procedure, evaluation, etc., can be associated with one or more codes, and there can be multiple coding schemes. In some cases, a researcher may be unaware of a complete list of codes that correspond to a particular treatment, condition, test, procedure, etc., that is of interest to the researcher. In some cases, codes may change over time and/or new codes may be added. For example, if a researcher wishes to conduct research that involves patients who presented with fever, the researcher may pose a question that simply uses the word “fever.” However, “fever” in health records may be coded in a variety of manners. For example, there are multiple ICD-10 codes for fevers of various origins or unknown origins (for example, the codes R50.2, R50.81, R50.82, R50.83, R50.84, and R50.9 all indicate fever). Even if the question is very specific, a health professional may not be aware of the corresponding codes. For example, a post-transfusion fever can be coded as R50.84, but the health professional may not be aware of this coding. Even if the researcher is aware of the specific code, it is possible that the physician or nurse who entered information into an EHR system used a different code instead, for example because they were not aware of the specific code. As another example, a health professional may be interested in those who have received a SARS-CoV-2 vaccination. SARS-CoV-2 vaccination can be associated with multiple CPT codes depending upon the particular vaccine, dosage, etc. A researcher may not be aware of all the specific codes for different vaccines, dosages, etc., and thus may not include all relevant codes when asking a question. Moreover, manually typing in codes can be a laborious and error-prone process. Automatically identifying relevant codes can save significant time and improve accuracy and completeness.

When searching health records, it can be important to use correct and complete codes to ensure that the correct patients are identified, the correct data is retrieved, and so forth. If codes are missing or incorrect, the data may be incomplete, unsuited to answering a question posed by a health professional, and so forth. Accordingly, there is a need for systems and methods that can receive a request from a health professional and determine appropriate codes based on the request. In some embodiments, a phenotype library can be used to map user-specified conditions, treatments, procedures, etc., to corresponding codes.

In some embodiments, a phenotype library is used to map conditions, treatments, etc., to appropriate codes. In some embodiments, a phenotype library can comprise a table that is used to store phenotype information. In some embodiments, a table maps keywords to codes. In some embodiments, codes are stored in a format that is compatible with a query language, such that the relevant syntax for a query can be extracted from the phenotype library without further transformation or modification. In some embodiments, the phenotype library includes textual descriptions. For example, if the keyword “cefpod” maps to ATC code J01DD64, the phenotype library can include a description of the code, such as “cefpodoxime and beta-lactamase inhibitor; systemic.” In some embodiments, the description is displayed to a user, which can help the user confirm that the correct codes are used. In some embodiments, the phenotype library includes a patient count, which can indicate the number of patients having at least one code for a phenotype associated with their medical records. In some embodiments, the phenotype library can include case count, which can indicate the number of times a particular phenotype has been used in a retrospective study. In some embodiments, the phenotype library stores an identifier for each retrospective study that used a particular phenotype.

Patient counts and case counts can be significant because, for example, they can be used to provide an initial indication of population size for a retrospective study or to indicate how often a particular phenotype is used in retrospective studies. A physician or other user can determine, based on the patient count, if the population size is likely to be too small to obtain reliable results. If so, the physician can modify the retrospective study definition, for example by revising a question or by requesting improvements from the system. In some embodiments, if a phenotype is rarely or never used for retrospective studies, this can indicate that the phenotype may not be well suited to retrospective studies. This can be especially useful when the physician or other user is researching a common health issue but may not provide a strong indication of usefulness of the phenotype when the researcher is studying a rare disease or other issue that is less commonly investigated.

As described herein, phenotypes can play an important role in determining the correct codes to use for a retrospective study. Thus, to obtain reliable results, it is important that the records in the phenotype library are correct. In some embodiments, the phenotype library can store approval information, such as approval status, an indication of who reviewed or approved the phenotype, an indication of who created the phenotype (which can be a person or algorithm), and so forth. In some embodiments, an approval status can be approved, not approved, or not reviewed. In some embodiments, only approved phenotypes are used in retrospective studies.

In some embodiments, a user interface is provided that allows users to approve or reject phenotypes. In some embodiments, the user interface is configured to allow users to modify, duplicate, or delete phenotype entries.

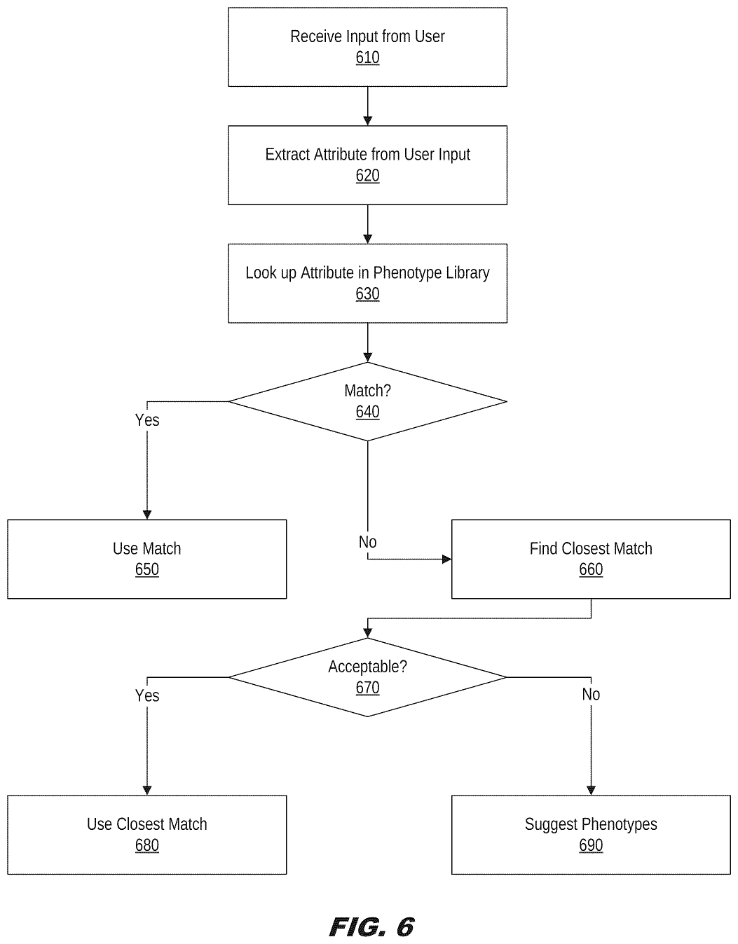

FIG. 6 is a flowchart that illustrates an example process for phenotyping according to some embodiments. The process illustrated in FIG. 6 can be carried out on a computer system.

At operation 610, the system can receive input from a user, for example in the form of a natural language question. The question can have one or more attributes, such as population, condition, treatment, outcome, and/or other parameters, for example timeframe. At operation 620, the system can extract an attribute from the user input. For example, in some embodiments, the system may generate a formatted question (e.g., a PICOT question) from a question submitted by a user as described above. At operation 630, the system can look up the attribute in a phenotype library. At decision 640, the system can determine if there is an exact match in the phenotype library. If so, the system can, at operation 650, use the exact match. If not, at operation 660, the system can find the closest match. For example, in some embodiments, a closest match can be based on a recommendation generated by an LLM. For example, in a PICOT, there may be an outcome such as “skeletal related event,” but the phenotype library may not contain a direct match for this term. In some embodiments, the LLM may identify related terms or concepts that do exist in the phenotype library, for example pathological fracture, spinal cord compression, etc. In some embodiments, the LLM may suggest these as related concepts. At decision 670, the system can determine if the closest match is acceptable (or if there is a closest match). For example, in some embodiments, the system may provide a user with a notification that there were no exact matches and may receive user input indicating that the closest match is or is not acceptable. In some embodiments, the system may return more than one match. For example, the system may return two closest matches, three closest matches, four closest matches, five closest matches, etc., and a user may select from the closest matches. If, at decision 670, the system determines that the closest match is acceptable (e.g., based on a user accepting the match), the system can, at operation 680, use the closest match for further processing. If, at decision 670, the closest match is not acceptable, the system can, at operation 690, generate a phenotype recommendation. For example, the system may suggest additional phenotypes or generate a new phenotype. In some embodiments, the system can generate a new phenotype using an LLM constrained using RAG performed on medical taxonomies.

In some embodiments, a phenotype library can include keywords, descriptions, corresponding query inputs, etc. In some embodiments, the phenotype library may include, for example, a number of patients matching the keyword, an indication of whether or not an entry has been reviewed or approved, a source of the entry, and so forth. As an example, an entry for a keyword “htn” may correspond to hypertensive disorders. The entry may have a description that includes words such as “hypertension” and/or “hypertensive.” In some embodiments, the description can be based on code descriptions, such as CPT code descriptions, ICD code descriptions, etc. In some embodiments, descriptions can, additionally or alternatively, be inferred based on patient medical records using a machine learning model such as a large language model. For example, descriptions can be determined by analyzing the contents of patient medical records such as narrative descriptions input by physicians, and common elements in the narrative descriptions can be used to generate a description for a phenotype.

As described herein, in some embodiments, a phenotype library may not have an exact match for a word, abbreviation, etc., entered by a user. In some embodiments, a large language model (LLM) can be used to find a closest matching entry in the phenotype library. However, it is known that in some cases, LLMs may hallucinate responses. While this may be inconsequential or of limited significance for some types of questions, it can be significant for others. For example, if an LLM hallucinates a phenotype that lists incorrect codes, irrelevant codes, etc., there can be significant impact on the study results. In some cases, such errors could lead to incorrect conclusions, improper treatment of patients, and so forth. Thus, it can be important to take measures to reduce or eliminate the likelihood of an LLM hallucinating an incorrect response.

In some embodiments, retrieval-augmented generation (RAG) can be used to constrain the results produced by an LLM. For example, the LLM may be constrained to providing phenotypes that already exist in a phenotype library. In some embodiments, the LLM can retrieve only the most relevant phenotypes selected from previously used phenotypes. RAG can provide an LLM with access to data outside of the data used to train the LLM.

In some embodiments, using a RAG approach, a system can be configured to locate and retrieve information relevant to a user's question. In some embodiments, the retrieved information can be appended to an LLM prompt, and the LLM can use the retrieved information as a basis for generating an answer to the prompt.

RAG is a technique that can enhance the capabilities of LLMs. Traditional LLMs are trained on large datasets that include a wide variety of subject matter. RAG can leverage external knowledge bases. When responding to a prompt or question, an LLM can retrieve relevant information from an external source (or another component can retrieve relevant information). The retrieved data can be incorporated into the internal processing of the LLM, enabling the LLM to produce results that are more accurate, factual, and/or contextually relevant. RAG can help to ground LLM responses in real-world knowledge.

For example, a system can receive an input query. The system can search an external knowledge base for documents or other information that is most relevant to the query. In some embodiments, the query and knowledge base content can be embedded into a common vector space, thereby enabling efficient retrieval based on semantic similarity. Once relevant documents or information are identified, RAG can use a ranking algorithm to determine the most informative or trustworthy sources. The ranking process can be influenced by, for example, document relevance, source credibility, publication date, etc. A subset of relevant documents can then be used for further processing. For example, the top document, top five documents, top ten documents, etc., can be used for further processing. Retrieved documents can be used to augment the original query. For example, key facts, summaries of relevant papers, etc., can be incorporated into the query to generate an enriched query. The enriched query can provide the LLM with context that can be useful in generating a response.

Physicians and other health professionals often use various shorthand and/or acronyms to describe treatments, conditions, procedures, etc. In some cases, a phenotype library may not have an entry with a keyword that corresponds to a word or words used in a question. In some embodiments, a system can be configured to use a machine learning model to predict codes that correspond to a user's input.

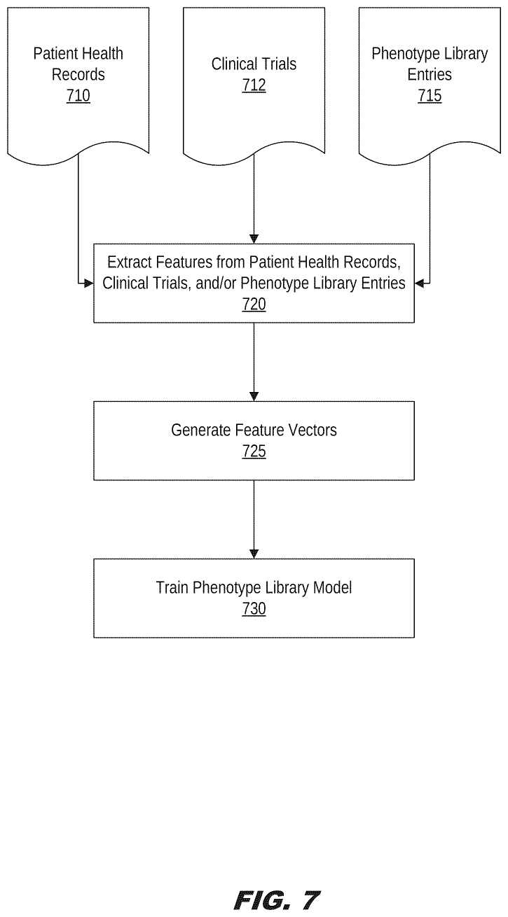

FIG. 7 illustrates an example of a process for training a machine learning model to generate phenotype library entries according to some embodiments. The process depicted in FIG. 7 can be performed by a computing system. In some embodiments, a system may generate phenotypes, but the phenotypes may not be added to a phenotype library or may not be available for use until reviewed. In some embodiments, a generated phenotype can be used for a current study, but may not be available for use in other studies.

At operation 720, the system can extract features from patient health records, clinical trials 712, phenotype library entries 715, or any combination thereof. At operation 725, the system can generate feature vectors from the extracted features. At operation 730, the system can train a phenotype library model to produce a trained model that can be used to generate new phenotype library entries from patient health records.

In some embodiments, training can be performed in a supervised manner. For example, given a set of patient health records 710, the expected outputs (phenotype library entries 715 or parts of phenotype library entries 715) can be known and the model can be trained so that the model produces outputs that closely or exactly match the expected outputs. That is, for example, a model can be trained using patient health records as input to produce already-known phenotypes. The model, once trained, can then be used to generate new phenotypes.

In some embodiments, a system can be configured to generate phenotypes. For example, phenotypes can be generated based on patient health records, past clinical trials, etc. As an example, if there are fifty known clinical trials for lung cancer, and many (e.g., a majority) of those clinical trials include a specific definition for a term such as “adverse event,” the system can extract the definition of “adverse event” from those clinical trials and add a coded representation of the definition to the phenotype library. In some embodiments, this can be done using a RAG system to identify common definitions across clinical trials.

In some embodiments, a system can generate phenotypes based on clinical terminologies as derived from sources such as the Unified Medical Language System (UMLS) or the Observational Medical Outcomes Partnership (OMOP). In some embodiments, a system can generate phenotypes by searching medical literature for existing studies related to a user's question.

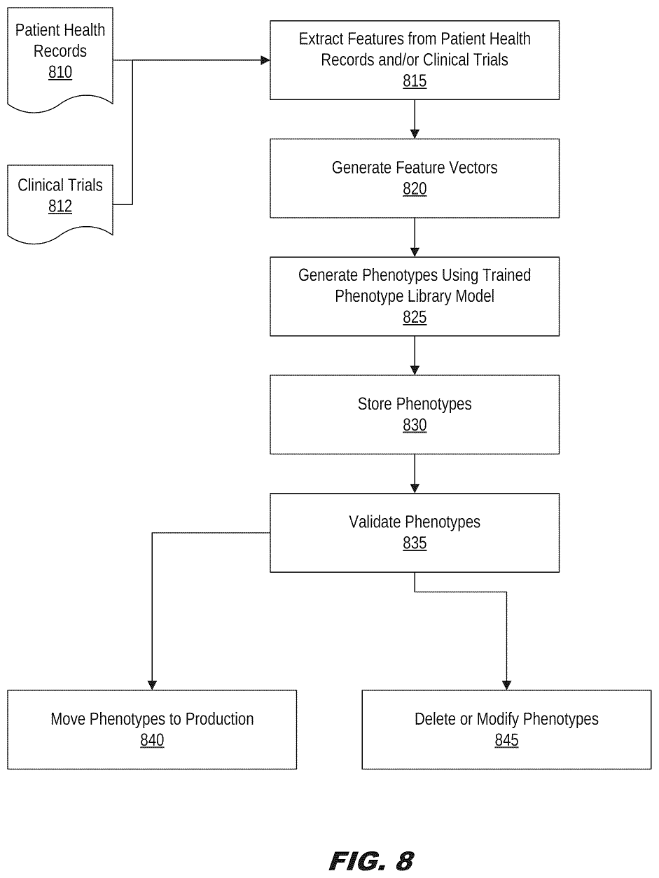

FIG. 8 illustrates an example process for generating phenotype library entries according to some embodiments. The process depicted in FIG. 8 can be performed on a computer system.

At operation 815, the system can extract features from patient health records 810, clinical trials 812, or both. The features can include, for example, patient demographics (e.g., age, sex, gender, height, weight), descriptions of patient encounters, treatment codes, procedure codes, diagnostic codes, definitions used in clinical trials, etc. As a non-limiting example, extracted features may include a list of codes associated with a patient and/or with a patient encounter, and medical terms, abbreviations, and so forth that appear in a physician's narrative about an encounter. At operation 820, the system can generate feature vectors that can be provided to a machine learning model (e.g., to a phenotype library model trained according to the process in FIG. 7). At operation 825, the system can generate phenotypes using the trained phenotype library model. At operation 830, the system can store the phenotypes. In some embodiments, generated phenotypes may be immediately available for use. In some embodiments, a generated phenotype may only be available for use after it is reviewed by a health professional who can verify the accuracy of the generated phenotype. In some embodiments, the system can perform an initial validation of the generated phenotype. For example, the system can determine a confidence in the generated phenotype, which can be provided to users. The confidence can be based on factors such as, for example, whether or not there are commonly-occurring comorbidities that could complicate phenotype generation, the number of patients records or unique patients involved in generating the phenotype, etc. At operation 835, the system can validate the generated phenotypes. For example, the system may provide a user interface accessible by physicians or other health professionals who can review the generated phenotypes to verify that they are accurate. In some embodiments the user interface may allow a reviewer to edit the generated phenotypes, for example to correct errors, to add additional information (e.g., to add additional codes, to add descriptions, etc.). If the reviewer accepts a generated phenotype at operation 835, the phenotype can be placed into production at operation 840 such that it can be used when users submit questions. If the reviewer rejects the phenotype at operation 835, the phenotype can be deleted at operation 845. In some embodiments, rather than deleting the phenotype, the phenotype may be flagged for further review, editing, and/or the like.

As an example, suppose a phenotype library lacks an entry for “hptn,” but the term “hptn” appears in patient health records that have ICD-10 codes of I10-I16 (e.g., essential (primary) hypertension, hypertensive heart disease, hypertensive chronic kidney disease, hypertensive heart and chronic kidney disease, secondary hypertension, and hypertensive crisis). The phenotype library model may process the patient health records and provide outputs indicating that the term “hptn” in patient encounter descriptions is commonly associated with the ICD-10 codes I10-I16, corresponding to hypertension.

In some embodiments, different approaches may be used. For example, in some embodiments, a system can extract medical terms, abbreviations, etc., from descriptions of patient encounters using, for example, a large language model. For example, a large language model can be provided with descriptions and corresponding medical terminology. The large language model can then be instructed to extract medical terminology from a provided description. In some embodiments the extracted medical terminology can have codes associated therewith (e.g., ICD codes, CPT codes, etc., that appear in the same encounter as the medical terminology). In some embodiments, a system can perform statistical analysis on the extracted terms, abbreviations, etc., to determine which codes are most strongly associated with which terms. For example, by analyzing records from many patients, frequency analysis can be used to determine which codes are most strongly associated with which terms.

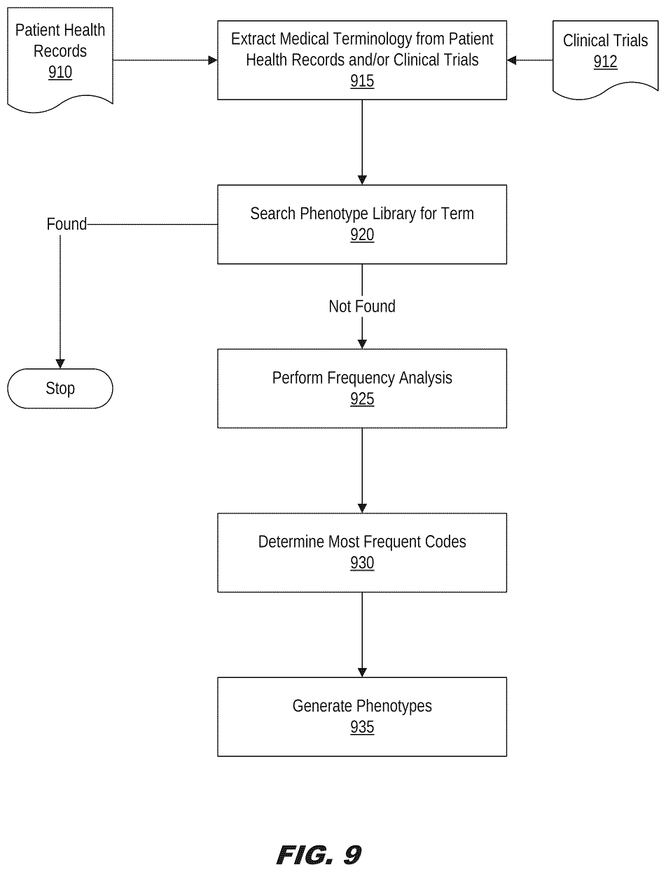

FIG. 9 is a flowchart that illustrates another example process for generating phenotypes according to some embodiments. The process illustrated in FIG. 9 can be performed on a computer system.

At operation 915, the system can extract medical terminology from patient health records 910, clinical trials 912, or both. For example, a language model (e.g., a large language model) can be used to extract encounter summaries from patient health records. The language model can then extract medical terminology from the extracted encounter summaries. For example, a language model might receive a summary such as “Pt has hx of hpn and presents with headache and elevated bp.” The language model may identify terms such as “hpn,” “headache,” and “elevated bp” as medical terms. As another example, clinical trials might use certain terminology and include definitions of certain terms, which can be extracted by the system. At operation 920, the system may search for each medical term in a phenotype library. If the term already exists in the phenotype library, the process can stop. For example, multiple terms may be used, and in some cases at least some of the terms may already exist in the phenotype library (e.g., “elevated bp” may already be defined in the phenotype library). If the term is not found, the system can perform frequency analysis at operation 925. The frequency analysis can include, for example, counts of how often particular codes appear in the medical history for a patient whose records include the term. In some embodiments, the frequency analysis can include a count of how often a particular code appears in a same record as the record in which the term appears. In some embodiments, the frequency analysis can include a count of patients for whom the term appears and the code appears in any part of the patients' medical records, regardless of whether or not the code and term appear as part of the same encounter. For example, a physician might note that a patient has hypertension, but the encounter may be unrelated to hypertension and codes for hypertension may not appear in the record for the encounter or may only be included as part of a patient history. At operation 930, the system can determine the most frequent codes for each medical term. For example, the most frequent codes for a patient with hypertension may include ICD-10 codes I10-I16, ATC/DDD codes beginning with C02, NDC codes corresponding to hypertension treatments, etc. At operation 935, based on the frequency analysis, the system can generate phenotypes that can be added to the phenotype library. As discussed above, in some embodiments, generated phenotypes may not be immediately available. For example, a generated phenotype may be available for use only after it is reviewed and approved.

While descriptions, summaries, etc., written by physicians and other health professionals often include information that can be used to generate phenotypes, other sources of information can also be used additionally or alternatively. For example, patients often undergo various forms of testing, such as bloodwork, urinalysis, fecal analysis, stress tests, sleep studies, imaging studies, and so forth. Test results can be indicative of a condition or conditions affecting the patient and can be used to help generate phenotypes. In some embodiments, lab results, information about prescription and/or non-prescription medications, information about procedures, and so forth can be used in generating phenotypes. For example, medications are often prescribed for the treatment of a specific condition or a limited set of conditions, and thus can be useful for determining phenotypes.

### Query Generation

In some embodiments, a phenotype library can be used to revise a formatted question (e.g., a PICOT) so that it uses relevant codes rather than natural language descriptions provided by the user. The PICOT can be used to generate a query to pull relevant data, calculate relevant statistics for a particular study, and so forth. For example, health record data may reside in a database or other data store. In some embodiments, the health record data may be organized in an in-memory datastore of patient objects. In some embodiments, health record data may be stored in an SQL database. In some embodiments, health record data may be stored in an object-oriented database, such as InterSystems Caché. Queries can be executed against a database to retrieve relevant records for analysis.

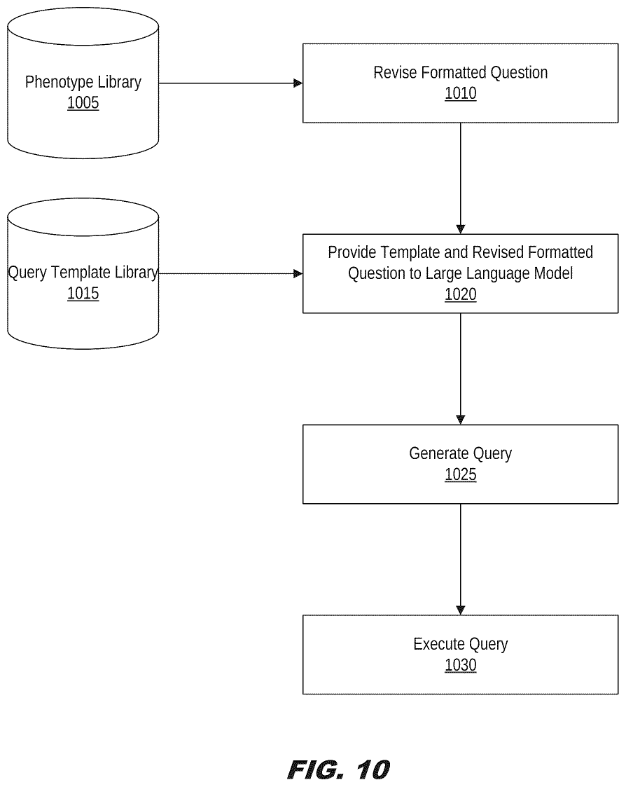

FIG. 10 illustrates an example process for generating a query according to some embodiments. The process depicted in FIG. 10 can be performed on a computer system. At operation 1010, the system can revise a formatted question (e.g., a PICOT). As shown in FIG. 10, the system can revise the formatted question using a phenotype library 1005, for example to convert text supplied by a user to relevant codes. At operation 1020, the system can select a template from a query template library 1015. The query template library 1015 can contain pre-written queries for different study types. The system can provide the template and the revised formatted question (e.g., revised PICOT) to a large language model (LLM). In some embodiments, the template may not be complete. In some embodiments, the template may not be executable. For example, the template may contain example queries, instructions for queries to generate, etc. As an example, a prompt submitted to an LLM could take a format such as “Generate a query for the following question: <modified question>. Here are three examples of queries: [example 1]; [example 2]; [example 3]. The query should be written in [language]. The database schema is: [schema].” In this example, <modified question> represents a version of a formatted question (e.g., PICOT) which includes relevant phenotypes, codes, etc., as they are represented in a database. [example #] represent examples of queries and can include descriptions of what queries represent, such as “select all patients between age 40 and 50 with phenotypes htn and coviddx: select * from patientRecords where age between 40 and 50 and phenotypes like ‘% htn %’ or phenotypes like %‘coviddx’.” [language] specifies the language for generating the query (e.g., SQL). [schema] describes the structure of the database(s) to be queried, such as table names, fields, types, primary keys, foreign keys, relations between tables, and so forth. In some embodiments, some information can be excluded, such as the language. For example, language may be inferred from the examples.

At operation 1025, the system can generate a query using the LLM. At operation 1030, the system can execute the query to retrieve results from a database.

In FIG. 10, a large language model is used to generate a query. However, in some embodiments, a large language model may not be used. For example, in some embodiments, a template may include executable query code with named fields in which information from the modified question (e.g., modified PICOT) can be substituted.

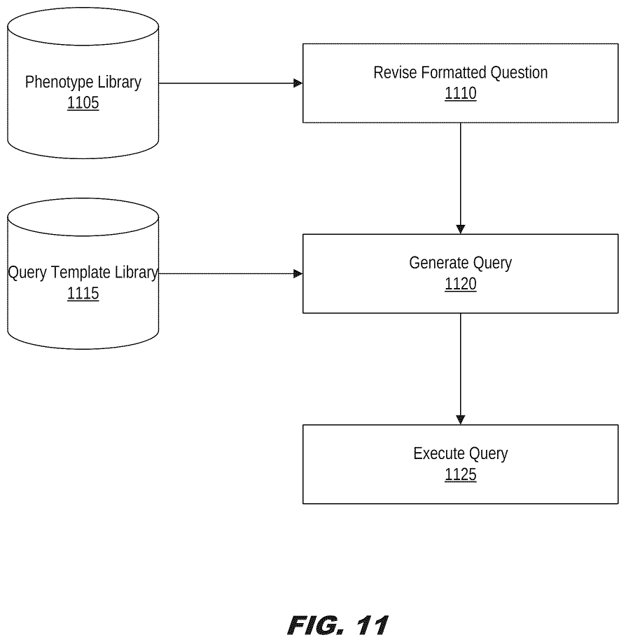

FIG. 11 is a flowchart that illustrates an example process for generating a query according to some embodiments. The process illustrated in FIG. 11 can be executed on a computer system. In FIG. 11, a template can be fully formed with fields that can be filled in based on the formatted question. For example, information from the formatted question can be plugged directly into the template or can be modified to be plugged into the template (e.g., into named fields within the template). A modification can be, for example, a data format conversion. For example, if a PICOT has a time range of “since 2005 Jan. 1” the system can convert this to “>=‘2005 Jan. 1’.” As another example, as described herein, terms that appear in the formatted question as presented to the user can be changed to phenotypes as defined in the phenotype library.

At operation 1110, the system can revise a formatted question (e.g., PICOT). For example, the system can revise the formatted question using the phenotype library 1105. As described above, the phenotype library can be used to convert text in the formatted question to relevant codes, phenotypes, etc. At operation 1120, the system can generate a query using the modified formatted question and a template selected from the query template library 1115. As mentioned above, in some embodiments, the template can be a template having named fields into which information from the modified formatted question can be substituted. At operation 1125, the system can execute the query to retrieve results from a database.

In some cases, a template may be a partial template that has some portions which can be filled in (e.g., named fields that can be filled in using information from a PICOT) and other portions that can be generated using a large language model. This might be the case if, for example, a PICOT or other formatted question includes information that is not templated in a form that the information can be readily plugged into.

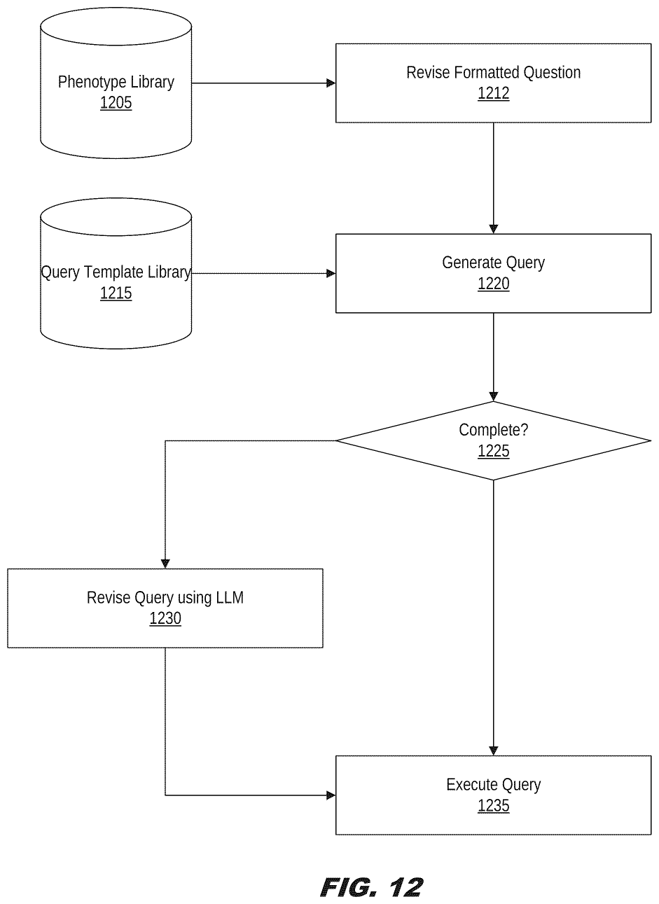

FIG. 12 is a flowchart that illustrates an example process for generating a query according to some embodiments. The process illustrated in FIG. 12 can be run on a computer system.

At operation 1210, the system can revise a PICOT using a phenotype library 1205, for example as described above. At operation 1220, the system can generate a query based on a template selected from the query template library 1215 and the revised PICOT. At operation 1220, the system may substitute named fields in the template retrieved from the query template library 1215 for information in the revised PICOT.

At operation 1225, the system can determine if the query is complete. For example, the system can determine if all named fields have been filled in, if all information from the formatted question (e.g., PICOT) has been included, etc. If the query is complete, the system can, at operation 1235, execute the query. If the query is not complete, the system can revise the query at operation 1230. For example, at operation 1230, the system may use the LLM to generate additional query code, for example to add information from the formatted question that was not substituted in at operation 1220. At operation 1235, the system can execute the query to retrieve relevant records from a database.

### Statistical Analysis and Data Summarization

Statistical analysis can produce large volumes of data that would be difficult for a physician or other health professional to digest quickly. In some cases, a physician or health professional may lack the requisite training or knowledge to understand the statistical analysis or may simply lack sufficient time to review the statistical analysis in detail. Many issues can arise if only raw statistics are provided. For example, insights may be missed, mistakes may be made in interpretation, and so forth. As just one example, a physician may not appreciate that a particular statistical result is not statistically significant.

In some embodiments, an automated pipeline can be used to perform statistical analysis. In some embodiments, a study template can be provided based on the type of study to be conducted. In some embodiments, study templates can, alternatively or additionally, be used to generate queries for retrieving data.

In some embodiments, various study types can be supported. For example, study designs can include observational studies and/or experimental studies/clinical trials. There can be various subtypes of studies. For example, experimental studies/clinical trials can include randomized controlled tests and/or non-randomized tests. Observational studies can include exploratory insight studies (e.g., case series, subgroup analysis) and/or causal comparative evidence studies (e.g., cohort studies (e.g., drug A vs. drug B), case-control studies (e.g., disease vs. healthy), differential outcomes studies, self-controlled studies (e.g., before/after), difference in differences studies, repeated measures studies, crossover studies, and so forth. In some embodiments, studies can be further broken down into, for example, odds ratio studies (e.g., binary outcome studies), Cohen's d studies, incident rate ratios, hazard ratio studies (e.g., survival outcomes), competing risks studies, time varying Cox proportional hazard studies, and so forth.

In some embodiments, statistics can include, for example, age distributions, race distributions, sex distributions, age-race distributions, age-sex distributions, race-sex distributions, distributions of intervention by race, age, sex, etc., mean age, median age, age standard deviation, mean pre-index days, mean follow-up days, number of encounters (mean and standard deviation), comorbidity scores (e.g., count, mean, standard deviation), outcome scores (e.g., mean, standard deviation) etc. For example, outcomes statistics may include the number and percent of patients who died, mean and standard deviation of days to death, days to related condition (e.g., a related injury or disease), and so forth. The particular statistics can vary from study to study and can depend upon, for example, PICOT inputs (e.g., there may not be a breakdown by sex if all patients were female, age range breakdowns may differ between a study of all adults and a study of only adults 65 and over, etc.).

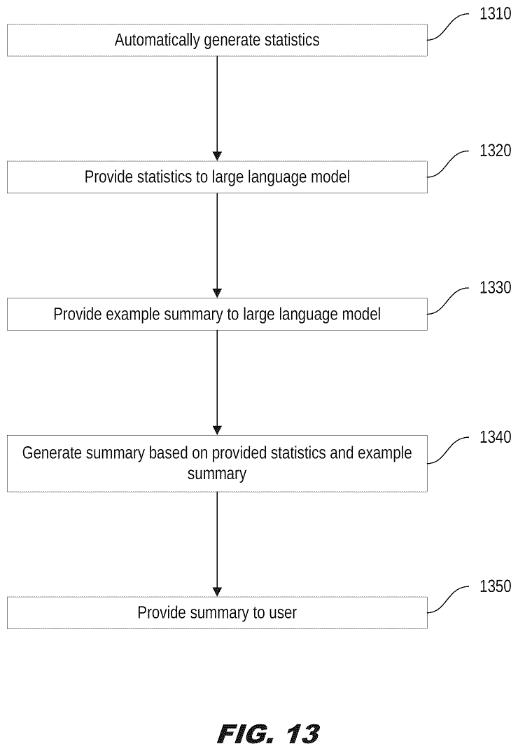

FIG. 13 is a flowchart that illustrates an example process for generating and summarizing statistical data according to some embodiments. The process depicted in FIG. 13 can be performed by a computer system.

At operation 1310, the system can automatically generate statistics. For example, as described herein, a template can specify particular statistics to be calculated. The statistics can be calculated based on the results of an executed query as described herein. At operation 1320, the system can provide statistics to an LLM. In some embodiments, a full statistical analysis may be provided to the LLM. However, in some embodiments, only select statistics may be provided to the LLM. For example, only statistics that should be included in a summary of the analysis may be included. In some embodiments, an LLM prompt can include, for example, instructions such as “Generate a scientific summary in about 100 words like the example summary below.” In some embodiments, the LLM prompt can include formatted data for inclusion in the summary. For example, the LLM prompt may include JSON-formatted data. In some embodiments, the data may include, for example, the question provided by the user, information about the study design (e.g., population, timeframe), and selected statistics to be included in the summary (e.g., number of patients, percent female, mean age, mean Charlson score, outcomes, etc.). In some embodiments, the LLM prompt may include an example summary. At operation 1330, the system can provide the example summary to the LLM. In some embodiments, operations 1320 and 1330 may be combined. For example, the statistics and example summary can be supplied in a same prompt. At operation 1340, the system can generate a summary based on the provided statistics, example summary, etc. At operation 1350, the system can provide the summary to a user. In some embodiments, the summary can be included in a report. In some embodiments, the report can include the summary and a full statistical analysis.

### Article Summarization

According to some embodiments as described herein, health professionals can pose natural language questions to a system and receive clinically relevant responses based on health record data. While such responses can be of immense value to health professionals, there may be articles (e.g., peer-reviewed, published studies) that can also be of interest or significance to a health professional. For example, published articles may reflect scenarios that are not reflected in the health records that were analyzed as part of a health record analysis and summarization process. This can be because, for example, published articles may discuss experimental treatment combinations, new drugs that are not in active use, different populations (e.g., different age ranges, different co-morbidities, etc.), and so forth.

As discussed herein, in some embodiments, a platform may have access to health records for a particular healthcare facility, healthcare system, etc. In some embodiments, multiple healthcare entities may share access to health record data, which can provide a more robust data set for answering questions submitted by health professionals. However, even if multiple healthcare entities share health record data, there can still be relevant data that is not accessible to the platform, and thus would be left out of statistical analyses performed using the platform.

While published articles can provide significant insights for health professionals, health professionals may struggle to locate and comprehend published articles. For example, there can be a large number of articles on any given topic, some or most of which are not especially relevant to the health professional's question. Even if a health professional locates potentially relevant articles, health professionals may not have time to read full articles to determine which are useful to the question or issue at hand. For example, physicians may be busy treating patients and have only limited time to read articles, and researchers may be busy performing experiments, analyzing experimental data, and so forth. Accordingly, there is a need for systems and methods that can locate relevant articles and provide high quality, factually sound summaries of the articles in a manner that health professionals can readily digest without undue time commitment.

In some embodiments, a system can be configured to retrieve articles that may be related to a question submitted by a user. For example, in some embodiments, a system can be configured to query published articles to identify articles that may be of interest. In some embodiments, a system can be configured to generate summaries of articles using a large language model.

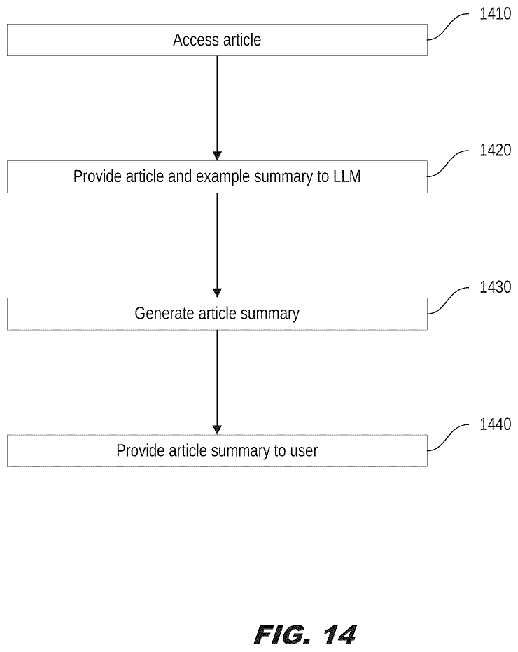

FIG. 14 is a flowchart that illustrates an example process for summarizing published articles according to some embodiments. The process depicted in FIG. 14 can be run on a computing system.

At operation 1410, the system can access an article. At operation 1420, the system can provide the article and example summary to an LLM. The system may include instructions for the LLM, such as “Generate a summary of the attached article in about 200 words, following the format of the example summary below.” At operation 1430, the system can generate an article summary using the LLM. At operation 1440, the system can provide the article summary to a user.

In some embodiments, the system may provide additional information, such as an estimate of the relevance of the article.

### ADDITIONAL EXAMPLES

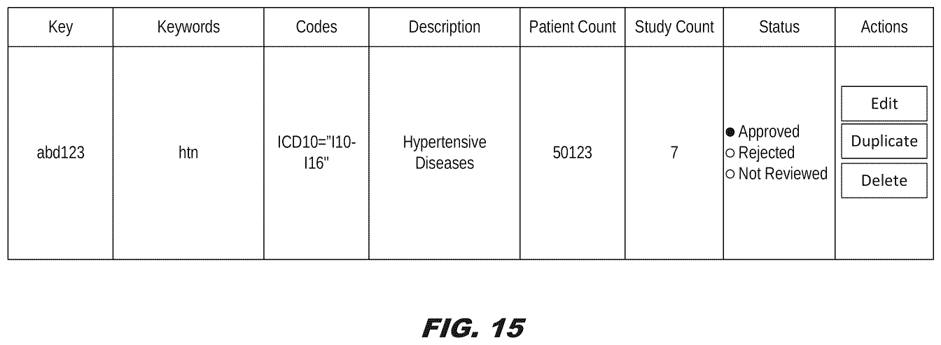

FIG. 15 is a schematic that illustrates an example of a phenotype database record and user interface for viewing phenotype database records according to some embodiments. A phenotype database can include various fields, such as a key (e.g., a unique identifier for each record in the phenotype database), keywords, codes (e.g., procedure codes, prescription codes, diagnosis codes, etc.), a description, patient count (e.g., number of patients whose records include the phenotype), study count (e.g., a number of prior studies that include the phenotype), and a status (e.g., approved, rejected, or not reviewed). As described herein, it is important that phenotypes be correct, as errors or omissions could lead to inaccurate or incomplete study results. Thus, in some cases, phenotype records may undergo one or more layers of review before being used in production. In some embodiments, as described herein, a machine learning model can generate phenotype records, for example based on analysis of patient health records. However, such phenotype records may not be available for use in production until an expert reviews them. In some embodiments, a reviewer can use the interface depicted in FIG. 15 to mark a phenotype record as approved, rejected, or needing further review. In some embodiments, when a machine learning model is used to generate a phenotype record, the phenotype record can be in a “not reviewed” state by default, and a reviewer can review the record and change its status to approved or rejected.

The user interface can include buttons or other user interface elements for interacting with phenotype records. For example, as shown in FIG. 15, a user can edit, duplicate, or delete a phenotype record. A user may edit a phenotype record to correct errors in, add information to, or delete information from a phenotype record. In some cases, a user may want to create a new phenotype record that is similar in one or more aspects to an existing record, in which case the user may duplicate the existing record and make modifications. In some cases, a phenotype record may be erroneous, outdated, or otherwise no longer needed, in which case a user may delete the record from the phenotype database.

In some embodiments, some fields in the phenotype database can be automatically generated. For example, in some embodiments, the “key” field can be a unique value that is automatically populated. In some embodiments, the patient count, study count, or both can be automatically populated based on the information in the codes field, keywords field, etc. For example, after a user (or machine learning model) defines the keywords and codes for a phenotype, a system can be configured to execute a query against past studies, patient health records, or both to determine appropriate values for the study count field, patient count field, or both.

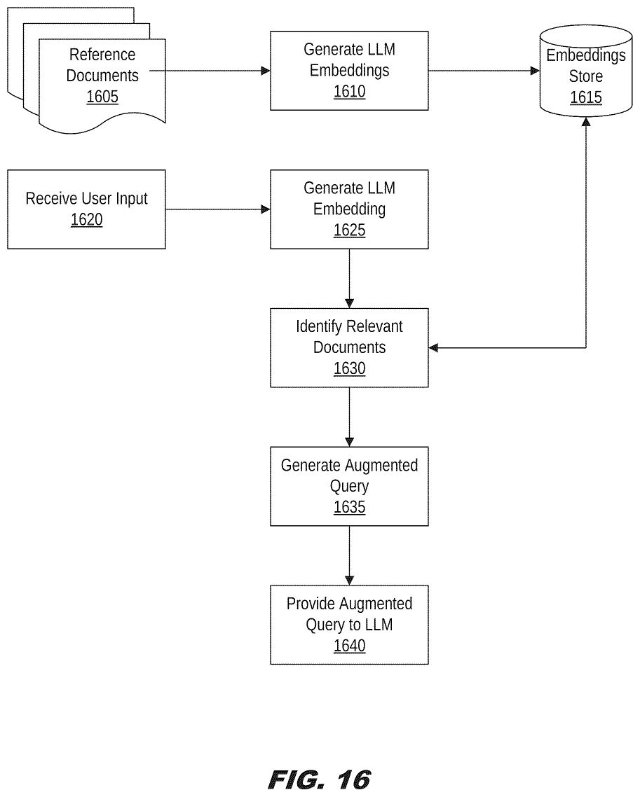

FIG. 16 is a flowchart that illustrates an example process for generated augmented queries using retrieval augmented generation according to some embodiments. At operation 1610, a system can generate LLM embeddings for reference documents 1605. The system can store the generated LLM embeddings in an embeddings store 1615. The embeddings store can be used to augment user inputs.

At operation 1620, the system can receive user input. At operation 1625, the system can generate an LLM embedding for the received user input. At operation 1630, the system can identify relevant reference documents. For example, the system can compare the generated LLM embedding for the received user input to the embeddings in the embeddings store and identify the embeddings in the embeddings store that are most similar to the embedding for received user input. Various metrics can be used to identify the most similar embeddings, such as cosine similarity, L1 distance, or L2 distance. At operation 1635, the system can generate an augmented query based on the received user input and the identified reference documents. In some embodiments, the system can extract context chunks from the identified reference documents, which can be appended to the user input to generate the augmented query. The augmented query can take various forms, but as one example, if a user input includes the term “nerve,” the user input can be augmented with information such as “Answer the question given that nerve relates to fibers that transmit electrical impulses between the brain and other parts of the body,” which can help address potential ambiguities such as interpreting “nerve” as courage or boldness. As another example, if a user input refers to “normal fasting glucose,” an augmented query can include information such as “Normal fasting glucose is defined as less than or equal to 100 mg/dL.” At operation 1640, the system can provide the augmented query to an LLM.

### Machine Learning

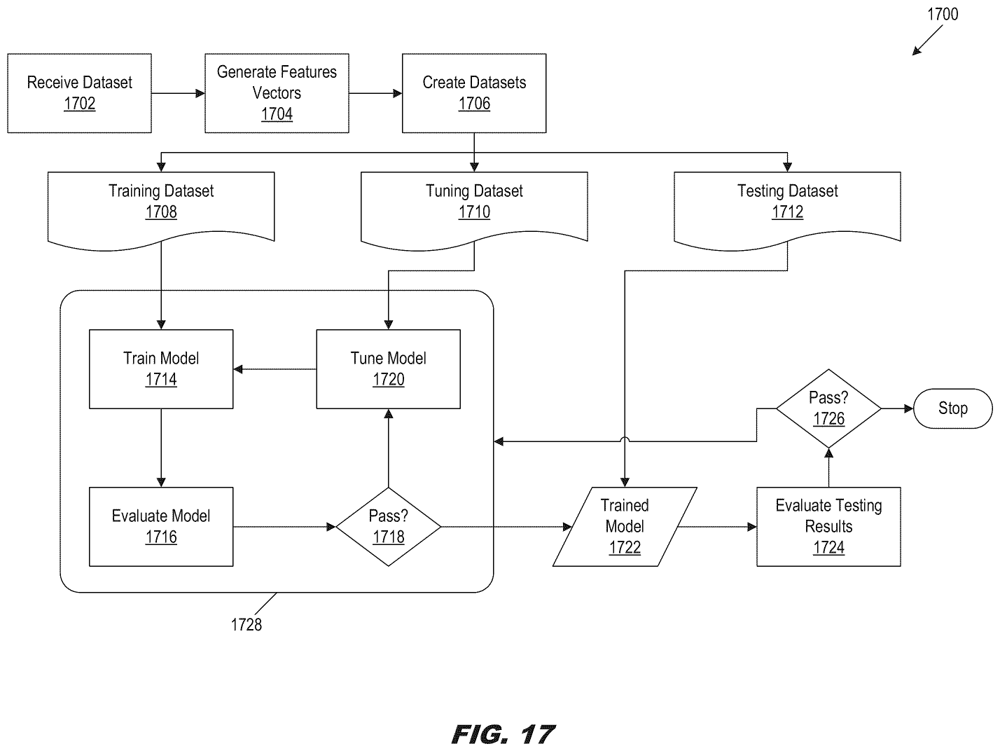

FIG. 17 depicts a process for training an artificial intelligence or machine learning model according to some embodiments. The process 1700 can be run on a computing system. At operation 1702, the system may access or receive a dataset. The dataset may include patient health records, journal articles, article summaries, clinical trial data, or any other relevant information. At operation 1704, the system can generate feature vectors based on the data set. Various processing steps can be performed when generating feature vectors. For example, the system may extract codes, lab results, vital signs, length of stay, demographic information, etc. In some embodiments, one or more transformations can be applied to the extracted data. For example, data may require transformations to conform to expected input formats, for example to conform with expected date and/or time formatting, to convert lab results to standard units, to convert temperatures to a common temperature scale, to convert weights to a common scale, and so forth. Strings and/or other information may be modified prior to use in machine learning training. For example, categorical data may be encoded in a particular manner. Nominal data may be encoded using one-hot encoding, binary encoding, feature hashing, or other suitable encoding methods. Ordinal data may be encoded using ordinal encoding, polynomial encoding, Helmert encoding, and so forth. Numerical data may be normalized, for example by scaling data to a maximum of 1 and a minimum of 0 or −1. These are merely examples, and the skilled artisan will readily appreciate that other transformations are possible. At operation 1706, the system may create, from the received dataset, training, tuning, and testing/validation datasets. The training dataset 1708 may be used during training to determine features for forming a predictive model. The tuning dataset 1710 may be used to select final models and to prevent or correct overfitting that may occur during training with the training dataset 1708, as the trained model should be generally applicable to a broad range of input data, not merely to data used for model training. The testing dataset 1712 may be used after training and tuning to evaluate the model. For example, the testing dataset 1712 may be used to check if the model is overfitted to the training dataset. The system, in training loop 1728, may train the model at operation 1714 using the training dataset 1708. Training may be conducted in a supervised, unsupervised, or partially supervised manner. At operation 1716, the system can evaluate the model according to one or more evaluation criteria. For example, the evaluation may include determining a false positive rate, false negative rate, true positive rate, true negative rate, and/or other metrics. At decision point 1718, the system may determine if the model meets the one or more evaluation criteria. If the model fails evaluation, the system may, at operation 1720, tune the model using the tuning dataset 1710, repeating the training operation 1714 and evaluation operation 1716 until the model passes the evaluation at decision point 1718. Once the model passes the evaluation at decision point 1718, the system may exit the model training loop 1728. The testing dataset 1708 may be run through the trained model 1722 and, at operation 1724, the system may evaluate the results. At decision point 1726, if the evaluation fails, the system may reenter training loop 1728 for additional training and tuning. If the model passes, the system may stop the training process, resulting in a trained model 1722. In some embodiments, the training process may be modified. For example, the system may not use a tuning dataset 1710. In some embodiments, the model may not use a testing dataset 1712.

### Computer Systems

Unless context indicates otherwise, methods described herein can be run on a single computer system or multiple computer systems. Computer systems can include physical computer systems, virtual computer systems (e.g., virtual machines), or both. The methods herein can, in some embodiments, be carried out in containers, which can act as a silo for running an application or applications on a host operating system. In some embodiments, a computer system can be headless and may not include a display device. In some embodiments, a computer system may not include or be connected (e.g., directly connected via a wired or wireless connection) to an input device such as a mouse or keyboard.

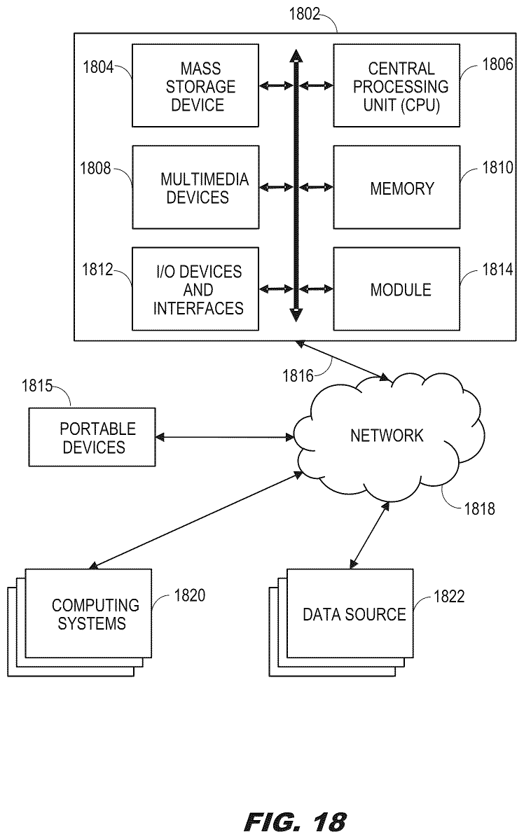

FIG. 18 is a block diagram depicting an embodiment of a computer hardware system configured to run software for implementing one or more embodiments disclosed herein.

In some embodiments, the systems, processes, and methods described herein are implemented using a computing system, such as the one illustrated in FIG. 18. The example computer system 1802 is in communication with one or more computing systems 1820 and/or one or more data sources 1822 via one or more networks 1818. While FIG. 18 illustrates an embodiment of a computing system 1802, it is recognized that the functionality provided for in the components and modules of computer system 1802 may be combined into fewer components and modules, or further separated into additional components and modules.

The computer system 1802 can comprise a module 1814 that carries out the functions, methods, acts, and/or processes described herein. The module 1814 is executed on the computer system 1802 by a central processing unit 1806 discussed further below.

In general, the word “module,” as used herein, refers to logic embodied in hardware or firmware or to a collection of software instructions, having entry and exit points. Modules are written in a program language, such as JAVA, C or C++, Python, or the like. Software modules may be compiled or linked into an executable program, installed in a dynamic link library, or may be written in an interpreted language such as BASIC, PERL, LUA, or Python. Software modules may be called from other modules or from themselves, and/or may be invoked in response to detected events or interruptions. Modules implemented in hardware include connected logic units such as gates and flip-flops, and/or may include programmable units, such as programmable gate arrays or processors.

Generally, the modules described herein refer to logical modules that may be combined with other modules or divided into sub-modules despite their physical organization or storage. The modules are executed by one or more computing systems and may be stored on or within any suitable computer readable medium or implemented in-whole or in-part within special designed hardware or firmware. Not all calculations, analysis, and/or optimization require the use of computer systems, though any of the above-described methods, calculations, processes, or analyses may be facilitated through the use of computers. Further, in some embodiments, process blocks described herein may be altered, rearranged, combined, and/or omitted.

The computer system 1802 includes one or more processing units (CPU) 1806, which may comprise a microprocessor. The computer system 1802 further includes a physical memory 1810, such as random-access memory (RAM) for temporary storage of information, a read only memory (ROM) for permanent storage of information, and a mass storage device 1804, such as a backing store, hard drive, rotating magnetic disks, solid state disks (SSD), flash memory, phase-change memory (PCM), 3D XPoint memory, diskette, or optical media storage device. Alternatively, the mass storage device may be implemented in an array of servers. Typically, the components of the computer system 1802 are connected to the computer using a standards-based bus system. The bus system can be implemented using various protocols, such as Peripheral Component Interconnect (PCI), Micro Channel, SCSI, Industrial Standard Architecture (ISA) and Extended ISA (EISA) architectures.

The computer system 1802 includes one or more input/output (I/O) devices and interfaces 1812, such as a keyboard, mouse, touch pad, and printer. The I/O devices and interfaces 1812 can include one or more display devices, such as a monitor, which allows the visual presentation of data to a user. More particularly, a display device provides for the presentation of GUIs as application software data, and multi-media presentations, for example. The I/O devices and interfaces 1812 can also provide a communications interface to various external devices. The computer system 1802 may comprise one or more multi-media devices 1808, such as speakers, video cards, graphics accelerators, and microphones, for example.

The computer system 1802 may run on a variety of computing devices, such as a server, a Windows server, a Structure Query Language server, a Unix Server, a personal computer, a laptop computer, and so forth. In other embodiments, the computer system 1802 may run on a cluster computer system, a mainframe computer system and/or other computing system suitable for controlling and/or communicating with large databases, performing high volume transaction processing, and generating reports from large databases. The computing system 1802 is generally controlled and coordinated by an operating system software, such as Windows XP, Windows Vista, Windows 7, Windows 8, Windows 10, Windows 11, Windows Server, Unix, Linux (and its variants such as Debian, Linux Mint, Fedora, and Red Hat), SunOS, Solaris, Blackberry OS, z/OS, iOS, macOS, or other operating systems, including proprietary operating systems. Operating systems control and schedule computer processes for execution, perform memory management, provide file system, networking, and I/O services, and provide a user interface, such as a graphical user interface (GUI), among other things.

The computer system 1802 illustrated in FIG. 18 is coupled to a network 1818, such as a LAN, WAN, or the Internet via a communication link 1816 (wired, wireless, or a combination thereof). Network 1818 communicates with various computing devices and/or other electronic devices. Network 1818 is communicating with one or more computing systems 1820 and one or more data sources 1822. The module 1814 may access or may be accessed by computing systems 1820 and/or data sources 1822 through a web-enabled user access point. Connections may be a direct physical connection, a virtual connection, and other connection type. The web-enabled user access point may comprise a browser module that uses text, graphics, audio, video, and other media to present data and to allow interaction with data via the network 1818.

Access to the module 1814 of the computer system 1802 by computing systems 1820 and/or by data sources 1822 may be through a web-enabled user access point such as the computing systems' 1820 or data source's 1822 personal computer, cellular phone, smartphone, laptop, tablet computer, e-reader device, audio player, or another device capable of connecting to the network 1818. Such a device may have a browser module that is implemented as a module that uses text, graphics, audio, video, and other media to present data and to allow interaction with data via the network 1818.

The output module may be implemented as a combination of an all-points addressable display such as a cathode ray tube (CRT), a liquid crystal display (LCD), a plasma display, or other types and/or combinations of displays. The output module may be implemented to communicate with input devices 1812 and they also include software with the appropriate interfaces which allow a user to access data through the use of stylized screen elements, such as menus, windows, dialogue boxes, tool bars, and controls (for example, radio buttons, check boxes, sliding scales, and so forth). Furthermore, the output module may communicate with a set of input and output devices to receive signals from the user.

The input device(s) may comprise a keyboard, roller ball, pen and stylus, mouse, trackball, voice recognition system, or pre-designated switches or buttons. The output device(s) may comprise a speaker, a display screen, a printer, or a voice synthesizer. In addition, a touch screen may act as a hybrid input/output device. In another embodiment, a user may interact with the system more directly such as through a system terminal connected to the score generator without communications over the Internet, a WAN, or LAN, or similar network.

In some embodiments, the system 1802 may comprise a physical or logical connection established between a remote microprocessor and a mainframe host computer for the express purpose of uploading, downloading, or viewing interactive data and databases on-line in real time. The remote microprocessor may be operated by an entity operating the computer system 1802, including the client server systems or the main server system, an/or may be operated by one or more of the data sources 1822 and/or one or more of the computing systems 1820. In some embodiments, terminal emulation software may be used on the microprocessor for participating in the micro-mainframe link.

In some embodiments, computing systems 1820 who are internal to an entity operating the computer system 1802 may access the module 1814 internally as an application or process run by the CPU 1806.

In some embodiments, one or more features of the systems, methods, and devices described herein can utilize a URL and/or cookies, for example for storing and/or transmitting data or user information. A Uniform Resource Locator (URL) can include a web address and/or a reference to a web resource that is stored on a database and/or a server. The URL can specify the location of the resource on a computer and/or a computer network. The URL can include a mechanism to retrieve the network resource. The source of the network resource can receive a URL, identify the location of the web resource, and transmit the web resource back to the requestor. A URL can be converted to an IP address, and a Domain Name System (DNS) can look up the URL and its corresponding IP address. URLs can be references to web pages, file transfers, emails, database accesses, and other applications. The URLs can include a sequence of characters that identify a path, domain name, a file extension, a host name, a query, a fragment, scheme, a protocol identifier, a port number, a username, a password, a flag, an object, a resource name and/or the like. The systems disclosed herein can generate, receive, transmit, apply, parse, serialize, render, and/or perform an action on a URL.

A cookie, also referred to as an HTTP cookie, a web cookie, an internet cookie, and a browser cookie, can include data sent from a website and/or stored on a user's computer. This data can be stored by a user's web browser while the user is browsing. The cookies can include useful information for websites to remember prior browsing information, such as a shopping cart on an online store, clicking of buttons, login information, and/or records of web pages or network resources visited in the past. Cookies can also include information that the user enters, such as names, addresses, passwords, credit card information, etc. Cookies can also perform computer functions. For example, authentication cookies can be used by applications (for example, a web browser) to identify whether the user is already logged in (for example, to a web site). The cookie data can be encrypted to provide security for the consumer. Tracking cookies can be used to compile historical browsing histories of individuals. Systems disclosed herein can generate and use cookies to access data of an individual. Systems can also generate and use JSON web tokens to store authenticity information, HTTP authentication as authentication protocols, IP addresses to track session or identity information, URLs, and the like.

The computing system 1802 may include one or more internal and/or external data sources (for example, data sources 1822). In some embodiments, one or more of the data repositories and the data sources described above may be implemented using a relational database, such as Sybase, Oracle, CodeBase, DB2, PostgreSQL, and Microsoft® SQL Server as well as other types of databases such as, for example, a NoSQL database (for example, Couchbase, Cassandra, or MongoDB), a flat file database, an entity-relationship database, an object-oriented database (for example, InterSystems Caché), a cloud-based database (for example, Amazon RDS, Azure SQL, Microsoft Cosmos DB, Azure Database for MySQL, Azure Database for MariaDB, Azure Cache for Redis, Azure Managed Instance for Apache Cassandra, Google Bare Metal Solution for Oracle on Google Cloud, Google Cloud SQL, Google Cloud Spanner, Google Cloud Big Table, Google Firestore, Google Firebase Realtime Database, Google Memorystore, Google MongoDB Atlas, Amazon Aurora, Amazon DynamoDB, Amazon Redshift, Amazon ElastiCache, Amazon MemoryDB for Redis, Amazon DocumentDB, Amazon Keyspaces, Amazon Neptune, Amazon Timestream, or Amazon QLDB), a non-relational database, or a record-based database.

The computer system 1802 may also access one or more databases 1822. The databases 1822 may be stored in a database or data repository. The computer system 1802 may access the one or more databases 1822 through a network 1818 or may directly access the database or data repository through I/O devices and interfaces 1812. The data repository storing the one or more databases 1822 may reside within the computer system 1802.

### Additional Embodiments

In the foregoing specification, the systems and processes have been described with reference to specific embodiments thereof. It will, however, be evident that various modifications and changes may be made thereto without departing from the broader spirit and scope of the embodiments disclosed herein. The specification and drawings are, accordingly, to be regarded in an illustrative rather than restrictive sense.

Indeed, although the systems and processes have been disclosed in the context of certain embodiments and examples, it will be understood by those skilled in the art that the various embodiments of the systems and processes extend beyond the specifically disclosed embodiments to other alternative embodiments and/or uses of the systems and processes and obvious modifications and equivalents thereof. In addition, while several variations of the embodiments of the systems and processes have been shown and described in detail, other modifications, which are within the scope of this disclosure, will be readily apparent to those of skill in the art based upon this disclosure. It is also contemplated that various combinations or sub-combinations of the specific features and aspects of the embodiments may be made and still fall within the scope of the disclosure. It should be understood that various features and aspects of the disclosed embodiments can be combined with, or substituted for, one another in order to form varying modes of the embodiments of the disclosed systems and processes. Any methods disclosed herein need not be performed in the order recited. Thus, it is intended that the scope of the systems and processes herein disclosed should not be limited by the particular embodiments described above.

It will be appreciated that the systems and methods of the disclosure each have several innovative aspects, no single one of which is solely responsible or required for the desirable attributes disclosed herein. The various features and processes described above may be used independently of one another or may be combined in numerous ways. All possible combinations and sub-combinations are intended to fall within the scope of this disclosure.

Certain features that are described in this specification in the context of separate embodiments also may be implemented in combination in a single embodiment. Conversely, various features that are described in the context of a single embodiment also may be implemented in multiple embodiments separately or in any suitable sub-combination. Moreover, although features may be described above as acting in certain combinations and even initially claimed as such, one or more features from a claimed combination may in some cases be excised from the combination, and the claimed combination may be directed to a sub-combination or variation of a sub-combination. No single feature or group of features is necessary or indispensable to each and every embodiment.

It will also be appreciated that conditional language used herein, such as, among others, “can,” “could,” “might,” “may,” “for example,” and the like, unless specifically stated otherwise, or otherwise understood within the context as used, is generally intended to convey that certain embodiments include, while other embodiments do not include, certain features, elements and/or steps. Thus, such conditional language is not generally intended to imply that features, elements and/or steps are in any way required for one or more embodiments or that one or more embodiments necessarily include logic for deciding, with or without author input or prompting, whether these features, elements and/or steps are included or are to be performed in any particular embodiment. The terms “comprising,” “including,” “having,” and the like are synonymous and are used inclusively, in an open-ended fashion, and do not exclude additional elements, features, acts, operations, and so forth. In addition, the term “or” is used in its inclusive sense (and not in its exclusive sense) so that when used, for example, to connect a list of elements, the term “or” means one, some, or all of the elements in the list. In addition, the articles “a,” “an,” and “the” as used in this application and the appended claims are to be construed to mean “one or more” or “at least one” unless specified otherwise. Similarly, while operations may be depicted in the drawings in a particular order, it is to be recognized that such operations need not be performed in the particular order shown or in sequential order, or that all illustrated operations be performed, to achieve desirable results. Further, the drawings may schematically depict one or more example processes in the form of a flowchart. However, other operations that are not depicted may be incorporated in the example methods and processes that are schematically illustrated. For example, one or more additional operations may be performed before, after, simultaneously, or between any of the illustrated operations. Additionally, the operations may be rearranged or reordered in other embodiments. In certain circumstances, multitasking and parallel processing may be advantageous. Moreover, the separation of various system components in the embodiments described above should not be understood as requiring such separation in all embodiments, and it should be understood that the described program components and systems may generally be integrated together in a single software product or packaged into multiple software products. Additionally, other embodiments are within the scope of the following claims. In some cases, the actions recited in the claims may be performed in a different order and still achieve desirable results.

Further, while the methods and devices described herein may be susceptible to various modifications and alternative forms, specific examples thereof have been shown in the drawings and are herein described in detail. It should be understood, however, that the embodiments are not to be limited to the particular forms or methods disclosed, but, to the contrary, the embodiments are to cover all modifications, equivalents, and alternatives falling within the spirit and scope of the various implementations described and the appended claims. Further, the disclosure herein of any particular feature, aspect, method, property, characteristic, quality, attribute, element, or the like in connection with an implementation or embodiment can be used in all other implementations or embodiments set forth herein. Any methods disclosed herein need not be performed in the order recited. The methods disclosed herein may include certain actions taken by a practitioner; however, the methods can also include any third-party instruction of those actions, either expressly or by implication. The ranges disclosed herein also encompass any and all overlap, sub-ranges, and combinations thereof. Language such as “up to,” “at least,” “greater than,” “less than,” “between,” and the like includes the number recited. Numbers preceded by a term such as “about” or “approximately” include the recited numbers and should be interpreted based on the circumstances (for example, as accurate as reasonably possible under the circumstances, for example ±5%, ±10%, ±15%, etc.). For example, “about 3.5 mm” includes “3.5 mm.” Phrases preceded by a term such as “substantially” include the recited phrase and should be interpreted based on the circumstances (for example, as much as reasonably possible under the circumstances). For example, “substantially constant” includes “constant.” Unless stated otherwise, all measurements are at standard conditions including temperature and pressure.

As used herein, a phrase referring to “at least one of” a list of items refers to any combination of those items, including single members. As an example, “at least one of: A, B, or C” is intended to cover: A, B, C, A and B, A and C, B and C, and A, B, and C. Conjunctive language such as the phrase “at least one of X, Y and Z,” unless specifically stated otherwise, is otherwise understood with the context as used in general to convey that an item, term, etc. may be at least one of X, Y or Z. Thus, such conjunctive language is not generally intended to imply that certain embodiments require at least one of X, at least one of Y, and at least one of Z to each be present. The headings provided herein, if any, are for convenience only and do not necessarily affect the scope or meaning of the devices and methods disclosed herein.

Accordingly, the claims are not intended to be limited to the embodiments shown herein but are to be accorded the widest scope consistent with this disclosure, the principles and the novel features disclosed herein.

What is claimed is:

1. A computer-implemented method for performing a retrospective medical study comprising:
receiving an input from a user via a user interface,
wherein the input comprises a natural language question,
wherein the input describes a medical research question;
processing the input to determine a study type for the input;
determining, based on the input, one or more study parameters,
wherein determining the one or more study parameters is done at least in part by providing the input to a first large language model;
generating, using the first large language model, a formatted study question based at least in part on the study type and the one or more study parameters;
processing the formatted study question to determine a phenotype associated with the formatted study question;
generating, based at least in part on the formatted study question and the phenotype, a database query;
executing the database query against a database to retrieve health record data for patients matching the one or more study parameters;
analyzing the retrieved health record data based on a predefined analysis procedure;
generating a summary of the analyzed health record data using a second large language model; and
making the summary available to the user.

2. The computer-implemented method of claim 1, wherein processing the input to determine the study type comprises identifying a specification of a study type in the input.

3. The computer-implemented method of claim 1, wherein processing the input to determine the study type comprises:
generating a prompt for the first large language model, the prompt comprising at least a portion of the input and an instruction to determine the study type;
submitting the prompt to the first large language model; and
receiving a response, the response comprising the study type.

4. The computer-implemented method of claim 1, further comprising:
identifying a publication related to the formatted study question;
accessing, over a communications network, an electronic version of the publication;
generating a summary of the publication using a third large language model; and
making the summary available to the user.

5. The computer-implemented method of claim 1, wherein the second large language model is the same as the first large language model.

6. The computer-implemented method of claim 1, wherein the first large language model is configured to use retrieval augmented generation to generate the formatted study question, wherein generating the formatted study question comprises:
generating an embedding of the input;
comparing the embedding of the input to a plurality of embeddings in a knowledge base;
identifying, based on the comparison, a set of relevant reference documents;
augmenting the input with contextual information derived from at least one of reference documents in the set of relevant reference documents; and
providing the augmented input to the first large language model.

7. The computer-implemented method of claim 6, wherein the input is augmented with a subset of the set of relevant documents, wherein the subset is determined by evaluating each reference document of the set of reference documents, wherein the evaluation is based at least in part on one or more of: relevance, source credibility, or publication date.

8. The computer-implemented method of claim 1, wherein the second large language model is configured to use retrieval augmented generation.

9. The computer-implemented method of claim 1, wherein determining the phenotype comprises:
identifying a term in the input indicating a condition, procedure, or medication;
querying a phenotype library using the identified term to identify a corresponding record; and
identifying one or more codes associated with the term.

10. The computer-implemented method of claim 1, further comprising, after generating the formatted study question:
providing the formatted study question to the user;
receiving a request from the user to revise the formatted study question; and
revising the formatted study question,
wherein revising the study question comprising modifying at least one of the one or more study parameters.

11. The computer-implemented method of claim 1, further comprising, after generating the formatted study question:
providing the study question to the user;
receiving a request to edit the formatted study question from the user;
receiving one or more user edits to the formatted study question; and
updating the formatted study question based on the one or more user edits.

12. The computer-implemented method of claim 1, wherein determining the phenotype comprises:
extracting a term from the input;
generate a prompt for a fourth large language model using the extracted term, the prompt configured to cause the fourth large language model to identify a closest matching phenotype;
provide the prompt to the fourth large language model;
receive an output from the fourth large language model responsive to the prompt; and
identify the phenotype based on the received output.

13. The computer-implemented method of claim 1, wherein analyzing the retrieved health record data based on the predefined analysis procedure comprises:
identifying a study template associated with the determined study type, wherein the study template includes one or more variable parameters;
updating at least one of the one or more variable parameters of the study template based on at least one of: the input, the one or more study parameters, or the phenotype; and
processing the updated study template to determine one or more variables of interest using the retrieved health record data.

14. The computer-implemented method of claim 1, wherein analyzing the retrieved health record data comprises:
determining a set of preliminary results;
providing the set of preliminary results to the user;
receiving a confirmation from the user to continue the analysis; and
in response to receiving the confirmation from the use, continuing the analysis of the retrieved health record data.

15. The computer-implemented method of claim 14, wherein the preliminary results comprise at least one of: number of a patients included in the health record data, age ranges of patients included in the health record data, number of patients receiving a particular treatment, or gender distribution of patients included in the health record data.

16. A system comprising:
at least one processor; and
a non-transitory, computer-readable medium having instructions stored thereon that, when executed by the at least one processor, cause the system to:
receive an input from a user via a user interface,
wherein the input comprises a natural language question, and
wherein the input describes a medical research question;
process the input to determine a study type for the input;
determine, based on the input, one or more study parameters, wherein determining the one or more study parameters is done at least in part by providing the input to a first large language model;
generate, using the first large language model, a formatted study question based at least in part on the study type and the one or more study parameters;
process the formatted study question to determine a phenotype associated with the formatted study question;
generate, based at least in part on the formatted study question and the phenotype, a database query;
execute the database query against a database to retrieve health record data for patients matching the one or more study parameters;
analyze the retrieved health record data based on a predefined analysis procedure;
generate a summary of the analyzed health record data using a second large language model; and
make the summary available to the user.

17. The system of claim 16, wherein the instructions are further configured to cause the system to:
generate a prompt for the first large language model, the prompt comprising at least a portion of the input and an instruction to determine the study type;
submit the prompt to the first large language model; and
receive a response, the response comprising the study type.

18. The system of claim 16, wherein the instructions are further configured to cause the system to:
identify a publication related to the formatted study question;
access, over a communications network, an electronic version of the publication;
generate a summary of the publication using a third large language model; and
make the summary available to the user.

19. The system of claim 16, wherein the first large language model is configured to use retrieval augmented generation to generate the formatted study question, wherein generating the formatted study question comprises:
generating an embedding of the input;
comparing the embedding of the input to a plurality of embeddings in a knowledge base;
identifying, based on the comparison, a set of relevant reference documents;
augmenting the input with contextual information derived from at least one of reference documents in the set of relevant reference documents; and
providing the augmented input to the first large language model.

20. The system of claim 16, wherein the instructions are further configured to cause to system to, after generating the formatted study question:
provide the formatted study question to the user;
receive a request from the user to revise the formatted study question; and
revise the formatted study question,
wherein revising the study question comprising modifying at least one of the one or more study parameters.
